SEQUENCE LISTING (15 sequences)

SEQ ID NO: 1 - DNA, 2631 bp, Homo Sapiens
ggatcctggacaggcaccccggcttggcgctgtctctccccctcggctcggagaggcccttcggcctgagggagcctcgccgcccgtccccggcacacgcgcacgcccggcctctcggcctctgccggagaaacaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtg

SEQ ID NO: 2 - protein, 769 aa, Homo Sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGAAPYLKTKFICVTPTTCSNTIDLPMSPRTLDSLMQFGNNGEGAEPSAGGQFESLTFDMELTSECATSPM

SEQ ID NO: 3 - DNA, 2303 bp, Homo Sapiens
ggatcctggacaggcaccccggcttggcgctgtctctccccctcggctcggagaggcccttcggcctgagggagcctcgccgcccgtccccggcacacgcgcacgcccggcctctcggcctctgccggagaaacaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggcgctgccccatacctgaagaccaagtttatctgtgtgacaccattcattgatgcagtttggaaataa

SEQ ID NO: 4 - protein, 720 aa, Homo Sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGAAPYLKTKFICVTFIDAVWK

SEQ ID NO: 5 - protein, 769 aa, Homo Sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDCTCILVSPLVYLYPDIPKEEAFGKYCRPESQEHPEADPGAAPYLKTKFICVTPTTCSNTIDLPMSPRTLDSLMQFGNNGEGAEPSAGGQFESLTFDMELTSECATSPM

SEQ ID NO: 6 - DNA, 20 bp, Artificial Sequence
  antisense sequence used to inhibit translation of endogenous Stat3 mRNA
actcaaactgccctcctgct

SEQ ID NO: 7 - DNA, 20 bp, Artificial Sequence
  antisense sequence used to inhibit translation of endogenous Stat3 mRNA
tctgaagaaactgcttgatt

SEQ ID NO: 8 - DNA, 19 bp, Artificial Sequence
  antisense sequence used to inhibit translation of endogenous Stat3 mRNA
gccacaatccgggcaatct

SEQ ID NO: 9 - DNA, 20 bp, Artificial Sequence
  antisense sequence used to inhibit translation of endogenous Stat3 mRNA
tggctgcagtctgtagaagg

SEQ ID NO: 10 - DNA, 20 bp, Artificial Sequence
  antisense sequence used to inhibit translation of endogenous Stat3 mRNA
tttctgttctagatcctgca

SEQ ID NO: 11 - DNA, 20 bp, Artificial Sequence
  antisense sequence used to inhibit translation of endogenous Stat3 mRNA
tagttgaaatcaaagtcatc

SEQ ID NO: 12 - DNA, 20 bp, Artificial Sequence
  antisense sequence used to inhibit translation of endogenous Stat3 mRNA
ttccattcagatcttgcatg

SEQ ID NO: 13 - DNA, 20 bp, Artificial Sequence
  antisense sequence used to inhibit translation of endogenous Stat3 mRNA
tctgttccagctgctgcatc

SEQ ID NO: 14 - DNA, 20 bp, Artificial Sequence
  antisense sequence used to inhibit translation of endogenous Stat3 mRNA
tcactcacgatgcttctccg

SEQ ID NO: 15 - DNA, 20 bp, Artificial Sequence
  antisense sequence used to inhibit translation of endogenous Stat3 mRNA
gagttttctgcacgtactcc

(19) United States
(12) Patent Application Publication
(10) Pub. No.: US 2009/0227489 A1
(43) Pub. Date: Sep. 10, 2009

(54) STAT3 Agonists and Antagonists and Therapeutic Uses Thereof

(75) Inventors: Hua Yu, Tampa, FL (US); Drew Pardoll, Brookeville, MD (US); Richard Jove, Tampa, FL (US); William Dalton, Temple Terrace, FL (US)

Correspondence Address: FISH & RICHARDSON, PC, P.O. BOX 1022, MINNEAPOLIS, MN, 55440-1022, US

(73) Assignees: Johns Hopkins University, Baltimore, MD (US); University of South Florida, Tampa, FL (US)

(21) Appl. No.: 12/420,704

(22) Filed: Apr. 8, 2009

Related U.S. Application Data
(63) Continuation of application No. 11/526,367, filed on Sep. 25, 2006, now abandoned.
Application No. 11/526,367 is a continuation of application No. 10/380,020, filed on Sep. 2, 2003, now abandoned.
(60) Provisional application No. 60/231,212, filed on Sep. 8, 2000.

(51) Int. Cl. A61K 38/02; A61K 31/7105; A61K 31/711

(52) U.S. Cl. 514/8; 514/44 A

(57) ABSTRACT

The present invention relates to methods for modulating, i.e., agonizing or antagonizing, Stat3 (Signal Transducer and Activator of Transcription3) signaling activity for use in gene therapy. Inhibition and/or activation of Stat3 signaling is an effective approach to modulate angiogenesis and the immune response for treatment and/or prevention of inflammation, infection, inflammation, immune disorders, and ischemia.

### CROSS-REFERENCES TO RELATED APPLICATIONS

This application is a continuation and claims the benefit of priority under 35 U.S.C. § 120 of U.S. application Ser. No. 11/526,367, filed Sep. 25, 2006, which is continuation of U.S. application Ser. No. 10/380,020, filed Sep. 2, 2003, now abandoned, which is a 35 U.S.C. § 371 national stage application of International Application PCT/US01/28254, filed Sep. 10, 2001, which claims priority under 35 U.S.C. § 119(e) to U.S. Provisional Application No. 60/231,212, filed Sep. 8, 2000, all of which are incorporated by reference in their entireties herein.

### STATEMENT AS TO FEDERALLY SPONSORED RESEARCH

The development of this invention was supported by grant numbers CA75243, CA55652 and CA77859 awarded by the National Institutes of Health. The Government therefore has certain rights in this invention.

### 1. INTRODUCTION

The present invention relates to methods for modulating, i.e., agonizing or antagonizing, Stat3 (signal transducer and activator of transcription3) signaling activity for use in gene therapy. Inhibition and/or activation of Stat3 signaling is an effective therapeutic approach to modulate angiogenesis and the immune-response in various diseases.

### 2. BACKGROUND OF THE INVENTION

Signal transducers and activators of transcription (STATs) are latent cytoplasmic transcription factors that function as intracellular effectors of cytokine and growth factor signaling pathways (Darnell, 1997, Science 277(5332):1630-1635). STAT proteins were originally defined in the context of normal cell signaling where STATs have been implicated in control of cell proliferation, differentiation, and apoptosis (Bromberg and Darnell, 2000, Oncogene, 19:2468-2473; Darnell et al., 1994, Science 264:1415-1421).

Stat3β is a dominant-negative Stat3 variant, which is a truncated form of Stat3 that contains the dimerization and DNA binding domain but lacks the transactivation domain (Catlett-Falcone et al., 1999, Immunity, 10:105-115). As a consequence, Stat3β can bind DNA but cannot transactivate gene expression, thus blocking Stat3 signaling in a trans-dominant negative fashion in most cases. Blocking Stat3 by Stat3β in U266 cells down-regulated expression of the Stat3-regulated Bcl-X_{L} gene, resulting in a dramatic sensitization of cells to Fas-mediated apoptosis in vitro (Catlett-Falcone et al., 1999, supra).

Effective gene therapy requires the killing of genetically untransduced cells (“bystander” cells) concomitant with genetically transduced cells. Because transfection efficiency is a rate-limiting step for gene therapy, the efficacy of cancer gene therapy is enhanced by bystander effects. The identification and characterization of specific molecules involved in Stat-mediated bystander effects could provide useful reagents and techniques for developing novel prophylactic and therapeutic methods.

Citation or discussion of a reference herein shall not be construed as an admission that such is prior art to the present invention.

### 3. SUMMARY OF THE INVENTION

The present invention provides methods for use of Stat3 agonists and antagonists for treatment of disclose involving angiogenesis and immune disorders. The invention is based, in part, on the Applicants' discovery that inhibition of Stat3 signaling results in the induction of a cascade of immunologic danger signals, which are normally produced only during inflammation and infection. Thus, the cellular expression of a Stat3 antagonist results in the production of soluble factors which can induce the expression of pro-inflammatory cytokines and chemokines in neighboring cells. The present invention takes advantage of this “bystander effect” of Stat3 activity modulators to provide methods and compositions for the treatment of a variety of conditions, diseases and disorders. The invention further provides methods for identification of such soluble factors, herein termed “immunological danger signals”. As used herein, the term “immunologic danger signals” refers to soluble factors produced as a result of inhibition of Stat3, which induce an immune response, such as a pro-inflammatory signal, e.g., a pro-inflammatory cytokine.

The present invention provides a method for modulating angiogenesis comprising administering to an individual in need of treatment an effective amount of a compound that agonizes or antagonizes the activity of Stat3.

The present invention further provides a method for the treatment or prevention of a hypoxic or ischemic condition or disorder, comprising administering to an individual in need of treatment an effective amount of a compound that increases the activity of Stat3, so that the hypoxic or ischemic condition or disorder is treated or prevented. In one embodiment, the compound is Stat3. In another embodiment, the compound is a constitutive active form of Stat3 (Stat3-C). In one embodiment, the compound is interleukin-6. In another embodiment, the condition or disorder is the result of ischemia, coronary-atherosclerosis, myocardial infarction, tissue ischemia in the lower extremities, infarction, inflammation, trauma, stroke, vascular occlusion, prenatal or postnatal oxygen deprivation, suffocation, choking, near drowning, carbon monoxide poisoning, smoke inhalation, trauma, including surgery and radiotherapy, asphyxia, epilepsy, hypoglycemia, chronic obstructive pulmonary disease, emphysema, adult respiratory distress syndrome, hypotensive shock, septic shock, anaphylactic shock, insulin shock, cardiac arrest, dysrhythmia, or nitrogen narcosis.

In one embodiment, a method is provided for the treatment or prevention of a proliferative angiopathy with neovascularization, comprising administering to an individual in need of treatment an effective amount of a compound that decreases the activity of Stat3, so that the a proliferative angiopathy is treated or prevented. In one embodiment, the proliferative angiopathy is diabetic microangiopathy. In another embodiment, the compound is Stat3β. In another embodiment, the compound is a negative regulatory protein. In another embodiment, the compound is a Stat3 antisense nucleic acid molecule. In yet another embodiment, the compound is a ribozyme specific to Stat3. In yet another embodiment, the compound is an inhibitor of a positive regulator of Stat3. In another embodiment, the compound is an antibody specific to Stat3.

The invention further provides a method for suppressing an immune response, comprising administering to an individual in need of treatment an effective amount of a compound that increases the activity of Stat3. In one embodiment, the compound is Stat3. In another embodiment, the compound is a constitutive active form of Stat3 (Stat3-C). In another embodiment, the compound is interleukin-6. In another embodiment, the treatment of the individual ameliorates a symptom of an autoimmune disease. In another embodiment, the autoimmune disease is insulin dependent diabetes mellitus, multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, idiopathic thrombocytopenia purpura, rheumatoid arthritis, cirrhosis, pemphigus vulgaris, autoimmune infertility, Goodpasture's disease, bullous pemphigoid, discoid lupus, ulcerative colitis, or dense deposit disease.

In another aspect of the invention, a method is provided for activating an immune response, comprising administering to an individual in need of treatment an effective amount of a compound that decreases the activity of Stat3, with the proviso that the treatment is not a cancer treatment. In one embodiment of this method, the compound is Stat3β. In another embodiment, the compound is a negative regulatory protein. In another embodiment, the compound is a Stat3 antisense nucleic acid molecule. In yet another embodiment, the compound is a ribozyme specific to Stat3. In another embodiment, the compound is an inhibitor of a positive regulator of Stat3. In another embodiment, the compound is an antibody specific to Stat3.

In various embodiments of the invention, the therapeutic compound is delivered via gene therapy. In alternative embodiments, the compound is delivered to the individual with a pharmaceutically acceptable carrier.

The invention further provides a method for identifying an immunologic danger signal comprising: (a) inhibiting Stat3 signaling activity in cells in culture; (b) separating the supernatant from said cells; (c) adding said supernatant, or fractions thereof, to immune cells; and (d) assaying for activation of said immune cells; such that if immune cells are activated by a cell supernatant or a fraction thereof, then an immunological danger signal is identified. The invention further provides a composition comprising the cell supernatant or a fraction thereof that is the product of this method.

In one embodiment of the method, the immune cells are macrophages. In a specific embodiment, said assaying for activation of said immune cells comprises assaying said macrophages for NO production. In another specific embodiment, said assaying for activation of said immune cells comprises assaying said macrophages for iNOS expression. In another specific embodiment, said assaying for activation of said immune cells comprises assaying said macrophages for RANTES expression.

In another embodiment, the immune cells of the method are neutrophils. In another embodiment, said assaying for activation of said immune cells comprises assaying said neutrophils for TNF-α expression. In another embodiment, the-immune cells are T cells. In a specific embodiment said assaying for activation of said immune cells comprises assaying said T cells for for IFN-γ expression. In another specific embodiment, said assaying for activation of said immune cells comprises assaying said T cells for IL-2 expression

In another embodiment, the cells of the method are B16 cells. In another embodiment, the Stat3 is suppressed by a Stat3 signaling activity antagonist. In another embodiment, the antagonist is a dominant negative Stat3 mutant. In yet another embodiment the antagonist is a negative regulatory protein. In another embodiment, the antagonist is a Stat3 antisense nucleic acid molecule. In yet another embodiment of the method, the antagonist is a ribozyme specific to Stat3. In another embodiment, the antagonist is an inhibitor of a positive regulator of Stat3. In another embodiment, the antagonist is an antibody specific to Stat3.

The following standard abbreviations are used herein: Stat, signal transducer and activator of transcription; Stat3, signal transducer and activator of transcription3; TRAIL, TNF-related apoptosis-inducing ligand; EMSA, electrophoretic mobility shift assay; hSIE, high-affinity sis-inducible element; EGFP, enhanced green fluorescence protein; FACS, fluorescence-activated cell sorting; pIRES, vector comprising an internal ribosome entry site; IL, interleukin; Stat3β or Stat3beta, a dominant negative form of signal transducer and activator of transcriptions; Stat3-C, a constitutive active form of signal transducer and activator of transcriptions; VEGF, vascular endothelial growth factor; pIRE, palindromic interferon response element.

### 4. BRIEF DESCRIPTION OF THE FIGURES

FIG. 1. Inhibition of endogenous Stat3 (SEQ. ID. NO: 1; SEQ. ID. NO:2) DNA-binding activity in B16 cells by overexpression of Stat3β (SEQ. ID. NO:3; SEQ. ID. NO:4). EMSA was performed with nuclear extracts prepared from B16 cells transfected with no DNA (lane 1), empty vector (lane 2) or Stat3β expression vector (lane 3). Extracts from NIH3T3 fibroblasts stimulated with EGF were used as a positive control for Stat1 and Stat3 (lane 4). Supershifts were performed using antibodies recognizing either Stat3 (α-ST3) or Stat3:3 (α-ST3β) with extracts derived from B16 cells transfected with no DNA (lanes 5-7), the empty vector (lanes 8-10), or Stat3β vector (lanes 11-13). ST3:3, and ST1:3, ST1:1 indicate migration of complexes containing Stat3 or Stat3β homodimers, Stat1/Stat3 heterodimers and Stat1/Stat1 homodimers, respectively. The asterisk indicates the position of supershifted complexes.

FIG. 2A-C. Soluble factors produced by Stat3β-transfected B16 cells induce growth inhibition of non-transfected B16 cells. A. Growth inhibition analysis using supernatants derived from either empty vector or Stat3β transfected B16 cells collected at 0 h, 12 h, 24 h, 36 h, 48 h after transfection. Growth inhibition of B16 cells by supernatants from Stat3β-B16 at various times was expressed as % inhibition based on the formula, (No. cells control−No. cells experimental)/No. cells control×100. For ³H-TdR incorporation assays, 0.25 μCi 3H-TdR was added during the last 4 h of incubation. For MTT assays, 5 μl MTT (10 mg/ml) was added during the last 4 h of incubation. B. Cell cycle analysis. B16 cells were transfected in the lower chambers of Transwell units. Five hours later, non-transfected B16 cells were added to upper chambers. Another 48 h later, B16 cells in the upper chambers were harvested for cell cycle analysis. C. Apoptosis assays. After incubating with transfected cells in the lower chambers for 48 h, cells in upper chambers were harvested and stained with Annexin V-PE and 7-AAD, followed by FACS analysis. FL2-H represents 7-AAD and FL3-H represents Annexin V-PE. These experiments (with the exception of MTT assays) were repeated at least three times with similar results.

FIG. 3A-C. Overexpression of Stat3β induces cell cycle arrest and apoptosis in B16 cells. A. Transfection efficiencies of pIRE-EGFP and PIRE-Stat3β in B16 cells as determined by GFP expression, and cell viability of B16 cells 48 h after transfection as determined by trypan blue exclusion. B. Cell cycle analysis was performed by propidium iodide staining at various times after transfection as indicated. G₀/G₁ phase arrest in Stat3β-transfected B16 cells was detected at 24, 36 and 48 h after transfection. C. 48 h after transfection, apoptosis was measured by Annexin V-PE staining followed by FACS analysis. Data shown represent one of three experiments with similar results.

FIG. 4. Stat3β overexpression in B16 cells results in induction of TRAIL mRNA expression. Ten μg of total cellular RNAs isolated from B16 (lanes 1 and 4), and B16 transfected with either pIRES-EGFP (lanes 2 and 5) or pIRES-Stat3β (lanes 3 and 6) were hybridized with each multiple probe before digestion with RNase. Separation of protected fragments was achieved by gel electrophoresis. Fragment assignment was determined by migration relative to internal standards. Induction of TRAIL RNA was confirmed by two additional RPA analyses.

FIG. 5A-B. Blocking Stat3 signaling in B16 cells stimulates production of soluble factors capable of inducing iNOS-dependent nitric oxide production by macrophages. A. Kinetics of availability of soluble factors after Stat3β transfection. Supernatants from transfected B16 cells were taken out at the times indicated. The data shown represent one of two experiments with similar results and expressed as μM nitrite ±SD, n=4. B. NO production by macrophages is iNOS-dependent. Data shown are representative of 5 independent experiments with similar results. S=supernatant; Mph=macrophage.

FIG. 6. Macrophages activated by the supernatant derived from Stat3β-transfected B16 cells confers NO-mediated cytostatic activity against untransfected B16 cells. Macrophages (˜1×10⁵) were incubated in 50% supernatants from Stat3β-transfected (S-Stat3β) or GFP-vector transfected (S-GFP) B16 cells for 6 h. The supernatants were replaced by normal complete medium and wild-type B16 cells (1×10⁴) were added. Cytostatic activity of macrophages is determined 48 h later and is expressed as % of inhibition of ³H-TdR incorporation. The data shown are the results of one of four similar experiments ±SD, n=4.

FIG. 7A-B. Stat3β expression in B16 cells upregulates the expression of pro-inflammatory chemokines and cytokines, which can stimulate peritoneal macrophages to produce NO. Elevated expression of pro-inflammatory cytokines and chemokines in B16 cells as a result of Stat3β expression (A. TNF-α, IL-6, IFN-β; B. IP-10). Total RNAs were prepared form mock-transfected, GFP-transfected, Stat3β-transfected and UV-irradiated B16 cells. Data shown have been confirmed with at least one more experiment, in which RNAs were prepared from independent transfectants and UV-irradiated B16 tumor cells.

FIG. 8A-B. Factors secreted by Stat3β-transfected B16 cells upregulate the expression of pro-inflammatory cytokines and chemokines by macrophages and neutrophils. Macrophages and neutrophils were incubated with supernatants derived from either empty vector-transfected, Stat3-transfected or UV-irradiated (macrophage only) B16 cells. A. RNAse protection assays using RNAs prepared from macrophages treated with various supernatants as indicated. Data shown represent RNAs pooled from two RNA preparations isolated from macrophages stimulated with supernatants derived from two independent transfections. B. TNF-α ELISA assays of neutrophils after incubating with supernatants or LPS as indicated. Two independent experiments were performed as shown. Levels of TNF-α secreted by neutrophils cultured in the supernatants derived from B 16-pIRES-EGFP transfectants were assigned as “1”.

FIG. 9A-B. Expression of Stat3β in tumor cells leads to activation of macrophages and T lymphocytes in vivo. Mice were injected with B16 cells (2×10⁶/mouse) transfected with either GFP or Stat3β expression vectors. Five days later, peritoneal macrophages and lymphocytes were tested for NO production and IFN-γ production, respectively. A. NO production by peritoneal macrophages. B. IFN-γ ELISPOT of lymphocytes.

FIG. 10A-B. Transfection of NIH3T3-Src cells with either: A. Stat3β expression vector; or B. Stat3 anti-sense oligos, results in reduced levels of VEGF protein as shown by Western blot. Src tyrosine kinase-mediated VEGF upregulation requires Stat3. Src tyrosine activity is known to upregulate VEGF expression. In Src-transformed NIH3T3 cells, VEGF expression is high.

FIG. 11. Expression of constitutively-activated Stat3 (SEQ. ID. NO:5) increases the production of VEGF in NIH3 fibroblasts. Left panel: Stat3 DNA-binding activity in NIH3T3 stable clones transfected with Stat3C, a mutant form of Stat3 that is constitutively activated. Nuclear extracts prepared from EGF induced NIH3T3 cells (EGF), the wild-type NIH3T3 cells (WT), and NIH3T3 stable clones transfected with Stat3C clones 1, 3, 6 and 7 (Stat3C-1, -3, -5, -6, -7, respectively) are incubated with the ³²P-labeled hSIE oligonucleotide probe and analyzed by EMSA. Right panel: Western blot analysis of VEGF protein levels in the WT and stable transfectants. β-actin is used as a standard to indicate the amount of protein loaded in each lane. Stat3C clones 1, 3, 6 and 7 had more Stat3 DNA-binding activity and higher levels of VEGF protein compared to those of wt NIH3T3 cells and Stat3C clone 5.

FIG. 12A-B. Blocking Stat3 signaling in tumor cells inhibits VEGF promoter activity. Both B16 (A) and SCK (B) murine tumor cells were transfected with constructs containing the luciferase cDNA in the absence (pluc) or presence of the VEGF promoter (VEGF). While VEGF promoter activity was high in both tumor cells, co-transfection with anti-sense oligonucleotides (ASO) against Stat3 resulted in a dramatic reduction of VEGF promoter activity.

FIG. 13. Inhibition of Stat3 signaling in tumor cells reduces the expression of the endogeneous VEGF gene. B16 tumor cells were transfected with either: A. Stat3β; or B. Stat3 anti-sense oligonucleotides (ASO) at 100 nM, 200 nM, or 300 nM. Western blot analyses indicated that a decrease in Stat3 protein is correlated with a reduction in VEGF protein. β-actin is used here to indicate the amount of protein loaded in each lane.

### 5. DETAILED DESCRIPTION OF THE INVENTION

The invention described in the subsections below provides therapeutic methods for modulating angiogenesis and the immune response by agonizing or antagonizing Stat3 signaling activity. Stat3 is an essential regulator of several cellular and physiological processes, such as cell cycle, apoptosis, the immune response, and angiogenesis, as exemplified by the experiments in Section 6, 7 and 8. Based on the discovery by the Applicants using a variety of approaches to modulate Stat3 activity, Stat3 activity modulators can up-regulate or down-regulate cell cycle, apoptosis, immune-response, and angiogenesis respectively. Accordingly, agonists and antagonists of Stat3 activity can be used to modulate cell cycle, apoptosis, immune-response, and angiogenesis to treat disorders involving dysfunctions of cell cycle, control of apoptosis, immune-response, and angiogenesis.

Such methods and compositions may be used to treat and/or prevent such diseases or disorders as, for example, ischemic diseases and proliferative angiopathies with neovascularization. The methods and compositions described herein may be used to augment the immuneresponse to treat various diseases, such as cancer or inflammatory diseases, or to suppress the immune response to treat diseases and disorders such as auto-immune disorders. Such target diseases and disorders are further described hereinbelow.

The invention provides methods of treatment and prophylaxis by administration to a subject of an effective amount of an agonist or antagonist of Stat3 activity, which are also referred to collectively herein as “Stat3 activity modulators” or “pharmaceuticals of the invention”. Such Stat3 activity modulators include, but are not limited to, peptides, polypeptides, nucleic acids, and small molecules. In particular, examples of polypeptide Stat3 activity modulators include, e.g., Stat3β, a dominant negative form of the Stat3 gene constitutive active Stat3, the wild-type Stat3 gene, product and antibodies specific to Stat3. Nucleotide sequences that can be used to inhibit Stat3 gene expression include, for example, antisense and ribozyme molecules, as well as gene or regulatory sequence replacement constructs designed to enhance the expression of Stat3, Stat3beta, or constitutive active Stat3 (e.g., expression constructs that place the Stat3 gene under the control of a strong promoter system). Such Stat3 activity modulators are described in detail hereinbelow.

The invention further provides methods for the identification of “immunologic danger signals” and compositions comprising such immunologic danger signals, which may be used to stimulate an immune response. As used herein, the term “immunologic danger signal” refers to a signal which stimulates an immune response, such as a pro-inflammatory signal, e.g. pro-inflammatory cytokines and chemokines. Such methods for identification of immunological danger signals are further described hereinbelow.

### 5.1 Methods for Treatment or Prevention of Ischemic Diseases

In one embodiment of the invention, methods are provided for stimulating angiogenesis using Stat3 agonists. The therapeutic effect of activating Stat3 signaling in this embodiment of the invention lies in the promotion of a) de novo formation of blood vessels, and b) sprouting from pre-existing vessels. In the context of this invention, both phenomenon will be jointly referred to as angiogenesis. The use of Stat3 agonists to promote angiogenesis may be used, for example, in preventing or treating ischemic diseases. Stat3 agonists may be administered to patients in need of such treatment to increase stimulated vessel growth, and consequentially increase tissue perfusion and blood flow, thereby overcoming the vascular insufficiency characteristic of ischemic diseases.

Gene therapy approaches and other pharmacological approaches, described in the subsections below, can be designed and used to augment Stat3 signaling with relation to the treatment of vascular insufficiencies.

5.1.1 Gene Therapy Approaches

Angiogenic molecules can be administered by way of gene transfer. With this strategy, the angiogenic protein, such as Stat3, a constitutive active form of Stat (Stat3-C), and agonists of Stat3 signaling, is delivered to the tissue in form of a nucleotide sequence encoding said protein. The gene can be delivered in an expression vector via a variety of approaches, including direct injection, electroporation, by way of transfected cells, or commercially available liposome preparations. The expression vector, usually consisting of a replication-deficient adenovirus, retrovirus, lentivirus, and/or an adeno-associated virus, is taken up by the host cells via receptor-mediated mechanisms and/or endocytosis (see 5.6.3).

The present invention relates to administering nucleotide sequences encoding constitutive active Stat3, agonists of Stat3, such as, but not limited to, interleukin-6 (IL-6), as well as the normal form of Stat3. A constitutive form of Stat3 is encoded by the Stat3-C mutant form of the Stat3 gene. In Stat3-C the substitution of two cysteine residues within the C-terminal loop of the SH2 domain of Stat3 produces a molecule that dimerizes spontaneously, binds to DNA, and activates transcription, thus giving rise to a constitutive active molecule (Bromberg et at., 1999, Cell 98:295-303).

Alternatively, replacing those tyrosine residues in STAT3 that are being phosphorylated upon activation with aspartic acid residues may result in a constitutive active molecule. Dependent on the molecular context, acidic amino acids such as aspartic acid can mimic a phosphate. As Stat3 is activated upon phosphorylation at said tyrosine residues, mimicking such phosphates constitutively by incorporation of an aspartic acid can render the molecule to be constitutively active. In order to replace the tyrosine residue in Stat3 with aspartic acid, site directed mutagenesis approaches which are well known to the skilled artisan can be used. The present invention also relates to the expression of proteins that activate Stat3, such as IL-6. Expression of said protein components via gene therapy and resulting activation of Stat3 can be used in order to promote angiogenesis in ischemic diseases.

Another embodiment of the invention relates to the expression of the normal form of the Stat3 protein component.

The nucleotide sequence to be expressed in a gene therapy approach has to be operatively linked to a promoter sequence. As it is known to the skilled artisan, enhancer/promoter sequences are essential for the expression of a given gene. Enhancer/promoter sequences also confer temporal and spatial regulation onto the expression pattern of a given gene.

Such enhancer/promoter sequences should be chosen dependent on the indicated disorder. In some cases tissue specific expression will be the preferred embodiment of the invention; in other cases systemic expression of the nucleotide sequence may be preferred. This decision will depend on the indicated disorder, and ultimately on the clinician. Expression specific to the tissue affected by vascular insufficiency or ischemia can be conferred by enhancer/promoter sequences that are active only in that tissue. Combining the right promoter sequences with the gene to be expressed will require some experimentation involving standard techniques known to the skilled artisan. In other disorders, inducible expression of the pro-angiogenic molecule, such as Stat3C, Stat3, or IL-6, may be indicated. As it is known to the skilled artisan, different enhancer/promoter sequences are active only in the absence and/or presence of a particular factor, which can be a metabolite, an anorganic molecule or a protein component. In the context of treating ischemic diseases, which are characterized by insufficient nutrient and oxygen supply of affected tissues, enhancer/promoter sequences that are induced upon hypoxia are the preferred embodiment of the invention.

Placing the expression of the nucleotide sequence of the invention under control of hypoxia constitutes a self-regulatory system. Once the oxygen concentration in the affected tissue falls below a certain threshold due to impaired blood-supply resulting from narrowed or blocked arteries, the expression of the angiogenic gene, i.e. Stat3, Stat3C or other constitutive forms of Stat3, IL-6, respectively, is up-regulated. The resulting newly formed vascular tissue provides an increased blood-flow in the affected tissue, thus increasing the oxygen concentration in said tissue. Consequently, the expression of the recombinant angiogenic gene will cease. This method ensures sufficient neovascularization but prevents vascular overgrowth that may be associated with too long exposure to or too high expression of angiogenic factors.

5.1.2 Pharmacological Approaches

Angiogenic molecules can be delivered by administering the recombinant proteins. Recombinant Stat3, Stat3-C, or IL-6, respectively, can be synthesized and purified as fusion proteins by recombinant DNA techniques. Fusing a “peptide tag” such as a polyhistidine tag, glutathione S-transferase (GST), or the E. coli maltose binding protein (MBP) to the angiogenic protein facilitates its purification. The fusion proteins can be synthesized in different host systems, such as, but not restricted to, bacteria, insects cells or mammalian cells. Methods of expressing said proteins in different systems and purifying them are described in section 5.6.2.

In another embodiment of the invention, the proteins can be immuno-purified using antibodies specific to the respective protein. An examplary approach comprises covalently linking antibodies specific to the protein which is to be purified to a solid matrix. Protein extracts of the host cells expressing the desired protein are added to the matrix under conditions that allow binding of said protein to the matrix via non-covalent binding to the antibodies. After contaminants have been removed by washing under suitable conditions, the protein can be eluted.

The recombinant proteins can then be administered by the drug delivery system of choice dependent on whether systemic or local administration of the protein is preferred for the treatment or prevention, respectively, of the indicated disorder. Various delivery systems are known and are described in section 5.6.4.

The administration of agonists of Stat3 signaling, such as but not limited too Stat3, Stat3-C, or IL-6 causes increased angiogenesis and subsequent increase in blood flow, thus restoring sufficient supply of nutrients and oxygen in the affected tissue.

5.2 Methods for Treatment or Prevention of Proliferative Angiopathies with Neovascularization

A plurality of disorders are caused by the overgrowth of blood vessels, herein referred to as proliferative angiopathies with neovascularization. An examplary disorder of this kind is diabetic microangiopathy with neovascularization. This disease is characterized by swollen retinal vessels that leak fluid as well an excess of retinal vessels which is diagnosed as diabetic retinopathy and can lead to blindness in the affected patients.

Because of the regulatory role of Stat3 in angiogenesis through its activating effect on VEGF, modulating the activity of Stat3 signaling is a target for treating diseases involving neovascularization. The presented invention relates to inhibiting angiogenesis in proliferative angiopathies with neovascularization (other than cancer) by reducing the activity of Stat3 signaling. The invention relates to inhibiting Stat3 using negative regulators of Stat3, such as a dominant negative form of Stat3, Stat3beta. The invention comprises inhibiting Stat3 using negative regulators of Stat3, such as SOCS and PIAS, inhibitors of Stat3 expression, such as antisense oligonucleotides and ribozymes, antibodies inhibitors of positive regulators of Stat3, such as inhibitors of the Src tyrosine kinase. The effects of the pharmaceuticals of the invention will be referred to in this context as antiangiogenic.

5.2.1 Gene Therapy Approaches

In its preferred embodiment, the Stat3 activity modulator is Stat3beta, a dominant negative form of Stat3. Compared to STAT3, STAT3beta lacks the C-terminal transactivation domain. STAT3beta fails to activate a pIRE containing promoter in transient transfection assays. Instead, co-expression of STAT3beta inhibits the transactivation potential of STAT3, thus effectively inhibiting Stat3 activity (Caldenhoven et al., 1996, Journal of Biological Chemistry 271:13221-13227). The dominant negative form of Stat3, Stat3beta, can be administered by a gene therapy approach as described 5.6.3. With this strategy, Stat3beta is delivered to the targeted tissue in form of a nucleotide sequence encoding Stat3beta under conditions that allow Stat3beta expression. In order for the Stat3beta gene to be expressed, the gene must be operatively linked to an enhancer/promoter sequence. In order to target only certain organs or tissues, tissue-specific and/or inducible enhancer/promoter sequences can be used. For a more detailed discussion of tissue-specific gene therapy see section 5.6.3.

Alternative embodiments of the inventions comprise other inhibitors of Stat3 signaling, such as, but not limited to, the SOCS negative regulatory molecules and the PIAS family of negative regulatory proteins (Starr and Hilton 1999, Bioessays 21:47-52). These factors can also be administered via gene therapy as described in 5.6.3. In order for these genes to be expressed, the respective gene must be operatively linked to an enhancer/promoter sequence. In order to target only certain organs or tissues, tissue-specific and/or inducible enhancer/promoter sequences can be employed.

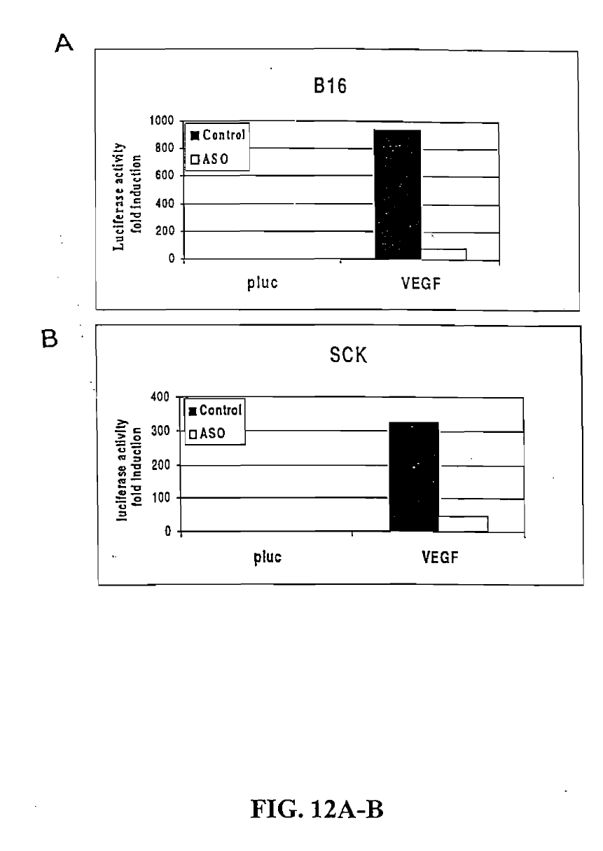

Additionally, the invention relates to suppressing the expression of endogenous Stat3. This can be achieved by administering nucleotide sequences that are in antisense orientation relative to the Stat3 encoding mRNA (hereinafter referred to as antisense Stat3 nucleotide sequence; see Example 3, FIG. 12). Those nucleotide sequences can vary in length from 20 basepairs up to the length of the entire Stat3 cDNA. Antisense nucleotide sequences of different length may differ in their efficacy as drugs, and it may take some experimentation to find the right length to treat the indicated disorder. Said antisense Stat3 nucleotide sequences can be delivered via gene transfer as described in 5.6.3. In order for these antisense nucleotide sequences to be expressed, the anitsense Stat3 nucleotide sequence must be operatively linked to an enhancer/promoter sequence. For targeting only certain organs or tissues, tissue-specific and/or inducible enhancer/promoter sequences can be employed.

Furthermore, expression of Stat3 can be suppressed by intracellular expression of small RNA therapeutics such as ribozymes. Small RNA therapeutics can be delivered via gene therapy by linking the nucleotide sequences encoding said RNA therapeutics opatively to an enhancer/promoter sequence. The invention encompasses the administration of a vector comprising the nucleotide sequence encoding the Stat3 specific ribozyme operatively linked to an enhancer/promoter to the patient by methods described in 5.6.3, thus resulting in an antiangiogenic effect.

5.2.2 Pharmacological Approaches

Anti-angiogenic molecules can also be delivered by administering the recombinant proteins. Recombinant Stat3beta, SOCS, or PIAS respectively, can be synthesized and purified as fusion proteins by recombinant DNA techniques. Fusing a “peptide tag” such as a polyhistidine tag, glutathione S-transferase (GST), or the E. coli maltose binding protein (MBP) to the angiogenic protein facilitates its purification. The fusion proteins can be synthesized in different host systems, such as, but not restricted to, bacteria, insects cells or mammalian cells. Alternatively the proteins can be immuno-purified using antibodies specific to the respective protein.

The recombinant proteins can then be administered by the drug delivery system of choice dependent on whether systemic or local administration of the protein is preferred for the treatment or prevention, respectively, of the indicated disorder. Various delivery systems are known and are described in 5.8.

Additionally, the invention relates to suppressing the expression of endogenous Stat3. This can be achieved by administering antisense Stat3 nucleotide sequences. Those nucleotide sequences can vary in length from 20 basepairs up to the length of the entire Stat3 cDNA. Antisense nucleotide sequences of different length may differ in their efficacy as drugs, and it may take some experimentation to find the right length to treat the indicated disorder. Said antisense Stat3 nucleotide sequences can be delivered by administering directly in vitro synthesized antisense nucleotide sequences. Those antisense nucleotide sequences can be modified to increase their stability, thus lengthening their half-life, in a cell. Antisense Stat3 nucleic acids are described in detailed in Section 5.6.4.

Furthermore, expression of Stat3 can be suppressed by administration of small RNA therapeutics such as ribozymes specific to Stat3 RNA. The invention comprises the in vitro synthesis of small RNA therapeutics such as ribozymes specific to Stat3 RNA and administration of said small RNA therapeutics. Those RNA therapeutics can be chemically modified in order to increase their stability and lengthen their half-life.

Furthermore, the invention relates to reducing neovascularization by antagonizing Stat3 signaling via inhibitors of positive regulators of Stat3 signaling such as the tyrosine kinase Src. In a specific embodiment, the invention encompasses the inhibition of Src by administration of the drug SU6656 (Blake et al. 2000, Molecular Cellular Biology 20:9018-9027).

The invention also comprises reducing neovascularization by antagonizing Stat3 signaling using antibodies specific to the Stat3 protein component. The antibodies can be administered by the drug delivery system of choice dependent on whether systemic or local administration of the protein is preferred for the treatment or prevention, respectively, of the indicated disorder. Various delivery systems are known and are described in 5.8.

### 5.3. Methods for Stimulating the Immune Response by Inhibiting Stat3 Signaling

In another embodiment, based on the regulatory effect of Stat3 on the production of such immunologic danger signals and the immune-response, the invention provides methods for stimulating the immune response using antagonists of Stat3 signaling activity. Immunologic danger signals are factors that attract cells of the immune-system to the site of the infection or cancerous growth and activate an immune response. Such immunologic danger signals include, but are not limited to: 1FN-gamma inducible protein 10 (IP-10), interleukin-6 (IL-6), tumor necrosis factor-alpha (TNF-alpha) and interferon-beta (IFN-beta).

In specific embodiments, the invention encompasses administration to a patient the supernatant of cells in which Stat3 activity is suppressed by means comprising Stat3beta and Stat3 antisense nucleotide sequences. The invention also encompasses inhibition of Stat3 signaling in the patient locally or systemically to augment the immune response in various diseases. The various embodiments of the invention are described in more detail in the sections below. The goal of any of these embodiments is to increase the concentration of immunologic danger signals either locally or systemically in the patient, thereby augmenting the immune response. Such a strengthening of the patient's own defense system is desirable when the patients natural immune reaction is not sufficient to eliminate the pathogen or the malignant cells. More specifically, some tumors evade immune surveillance by suppressing the expression of said immunologic danger signals.

5.3.1. Approaches for Administering the Supernatant of Stat3Beta Transfected Cells

This embodiment of the invention relates to the inhibition of Stat3 signaling in cells such as B16 melanoma cells by such means as expression of Stat3beta, expression of negative regulators of Stat3 signaling as for example PIAS and SOCS, expression of Stat3 antisense nucleotide sequences, administration of in vitro synthesized Stat3 antisense nucleotide sequences, and antibodies specific to Stat3. In the preferred embodiment of the invention, Stat3beta is expressed in B16 melanoma cells by means of transfection and supernatant is obtained from said cell culture. For a detailed description of the methods involved refer to Example 2 and sections 5.6.1 and 5.6.2.

The supernatant can then be administered to a patient in order to augment the immune response in various diseases. Such diseases include infectious diseases and various malignancies. The supernatant can be administered by any method known in the art. Some examples of which are described in section 5.6.4. Said supernatant can be converted into solid form by means such as to lyophilization

5.3.2 Gene Therapy Approaches to Augment the 17vIMLINE-Response in Various Diseases Other than Cancer

In a preferred embodiment, the pharmaceutical of the invention is Stat3beta, a dominant negative form of Stat3. Compared to STAT3, STAT3beta lacks the C-terminal transactivation domain. STAT3beta fails to activate a pIRE containing promoter in transient transfection assays. Instead, co-expression of STAT3beta inhibits the transactivation potential of STAT3, thus effectively inhibiting Stat3 activity (Caldenhoven et al. 1996, Journal of Biological Chemistry 271: 13221-13227). The dominant negative form of Stat3, Stat3beta, can be administered by a gene therapy approach as described 5.6.3. With this strategy, Stat3beta is delivered to the targeted tissue in form of a nucleotide sequence encoding Stat3beta under conditions that allow Stat3beta expression. In order for the Stat3beta gene to be expressed, the gene must be operatively linked to an enhancer/promoter sequence. In order to target only certain organs or tissues, tissue-specific and/or inducible enhancer/promoter sequences can be used. For a more detailed discussion of tissue-specific gene therapy see section 5.6.3.

Alternative embodiments of the inventions comprise other inhibitors of Stat3 signaling, such as, but not limited to, the SOCS negative regulatory molecules and the PIAS family of negative regulatory proteins (Starr and Hilton 1999, Bioessays 21:47-52). These factors can also be administered via gene therapy as described in 5.6.3. In order for these genes to be expressed, the respective gene must be operatively linked to an enhancer/promoter sequence. In order to target only certain organs or tissues, tissue-specific and/or inducible enhancer/promoter sequences can be employed.

Additionally, the invention relates to suppressing the expression of endogenous Stat3. This can be achieved by administering nucleotide sequences that are in antisense orientation relative to the Stat3 encoding mRNA (hereinafter referred to as antisense Stat3 nucleotide sequence; see Example 3, FIG. 12). Those nucleotide sequences can vary in length from 20 basepairs up to the length of the entire Stat3 cDNA. Antisense nucleotide sequences of different length may differ in their efficacy as drugs, and it may take some experimentation to find the right length to treat the indicated disorder. Such antisense Stat3 nucleotide sequences can be delivered via gene transfer as described in 5.6.3. In order for these antisense nucleotide sequences to be expressed, the antisense Stat3 nucleotide sequence must be operatively linked to an enhancer/promoter sequence. For targeting only certain organs or tissues, tissue-specific and/or inducible enhancer/promoter sequences can be employed.

Furthermore, expression of Stat3 can be suppressed by intracellular expression of small RNA therapeutics such as ribozymes. Small RNA therapeutics can be delivered via gene therapy by linking the nucleotide sequences encoding RNA therapeutics operatively to an enhancer/promoter sequence. The invention encompasses the administration of a vector comprising the nucleotide sequence encoding the Stat3 specific ribozyme operatively linked to an enhancer/promoter to a patient by methods described in 5.6.3, thus enhancing the immune response of the patient.

5.3.3 Pharmacological Approaches to Augment the Immuno Response in Various Diseases Other than Cancer

Antagonists of Stat3 signaling activity can be delivered by administering the recombinant proteins. Recombinant Stat3beta, SOCS, or PIAS respectively, can be synthesized and purified as fusion proteins by recombinant DNA techniques. Fusing a “peptide tag” such as a polyhistidine tag, glutathione S-transferase (GST), or the E. coli maltose binding protein (MBP) to the protein of the invention facilitates its purification. The fusion proteins can be synthesized in different host systems, such as, but not restricted to, bacteria, insects cells or mammalian cells. Alternatively the proteins can be immune-purified using antibodies specific to the respective protein.

The recombinant proteins can then be administered by the drug delivery system of choice dependent on whether systemic or local administration of the protein is preferred for the treatment or prevention, respectively, of the indicated disorder. Various delivery systems are known and are described in 5.8.

Additionally, the invention relates to suppressing the expression of endogenous Stat3. This can be accomplished by administering antisense Stat3 nucleotide sequences. Those nucleotide sequences can vary in length from 20 basepairs up to the length of the entire Stat3 cDNA. Antisense nucleotide sequences of different lengths may differ in their efficacy as drugs, and it may take some experimentation to find the right length to treat the indicated disorder. Said antisense Stat3 nucleotide sequences can be delivered by administering directly in vitro synthesized antisense nucleotide sequences. Those antisense nucleotide sequences can be modified to increase their stability, thus lengthening their half-life in a cell.

Furthermore, expression of Stat3 can be suppressed by administration of small RNA therapeutics such as ribozymes specific to Stat3 RNA. The invention comprises the in vitro synthesis of small RNA therapeutics such as ribozymes specific-to-Stat3 RNA and administration of said small RNA therapeutics. Those RNA therapeutics can be chemically modified in order to increase their stability and lengthen their half-life.

Furthermore, the invention relates to enhancing the immune response by antagonizing Stat3 signaling via inhibitors of positive regulators of Stat3 signaling such as the tyrosine kinase Src. In a specific embodiment, the invention encompasses the inhibition of Src by administration of the drug SU6656 (Blake et al. 2000, Molecular Cellular Biology 20:9018-9027).

The invention also comprises augmenting the immune response by antagonizing Stat3 signaling using antibodies specific to the Stat3 protein. The antibodies can be administered by the drug delivery system of choice dependent on whether systemic or local administration of the protein is preferred for the treatment or prevention, respectively, of the indicated disorder. Various delivery systems are known and are described in 5.8.

### 5.4. Methods for Inhibiting the Immune Response by Activating Stat3 Signaling

In another embodiment, based on the regulatory effect of Stat3 on the production of such immunologic danger signals and the immune-response, the invention provides methods for inhibiting the immune response using agonists of Stat3 signaling activity. Immunologic danger signals are factors that attract cells of the immune-system to the site of the infection or transplants and activate an immune response. Such immunologic-danger signals include, but are not limited to: IFN-gamma inducible protein 10 (IP-10), interleukin-6 (IL-6), tumor necrosis factor-alpha (TNF-alpha) and interferon-beta (IFN-beta).

In its specific embodiments, the invention encompasses administration to a patient of agonists of Stat3 signaling locally or systemically to suppress the immune response in various diseases. The various embodiments of the invention are described in more detail in the sections below. The goal of any of these embodiments is to decrease the concentration of immunologic danger signals either locally or systemically in the patient, thereby suppressing the immune response. Such a suppression of the patient's own defense system is desirable when the patient is suffering from autoimmune diseases or to ameliorate adverse reactions to transplants.

5.4.1 Gene Therapy Approaches to Suppress the Immune-Response in Various Diseases

Immuno suppressant molecules can be administered by way of gene transfer. With this strategy, the immuno suppressant protein, such as Stat3, a constitutive active form of Stat (Stat3-C), and agonists of Stat3 signaling, is delivered to the-tissue in form of a nucleotide sequence encoding said protein. The gene can be delivered in an expression vector via a variety of approaches, including direct injection, electroporation, by way of transfected cells, or commercially available liposome preparations. The expression vector, usually consisting of a replication-deficient adenovirus, retrovirus, lentivirus, and/or an adeno-associated virus, is taken up by the host cells via receptor-mediated mechanisms and/or endocytosis.

The present invention relates to administering nucleotide sequences encoding constitutive active Stat3, agonists of Stat3, such as, but not limited to, interleukin-6 (IL-6), as well as the normal form of Stat3. A constitutive form of Stat3 is encoded by the Stat3-C mutant form of the Stat3 gene. In Stat3-C the substitution of two cysteine residues within the C-terminal loop of the SH2 domain of Stat3 produces a molecule that dimerizes spontaneously, binds to DNA, and activates transcription, thus giving rise to a constitutive active molecule (Bromberg et al., 1999, Cell 98:295-303).

Alternatively, replacing those tyrosine residues in STAT3 that are being phosphorylated upon activation with aspartic acid residues may result in a constitutive active molecule. Dependent on the molecular context, acidic amino acids such as aspartic acid can mimic a phosphate. As Stat3 is activated upon phosphorylation at said tyrosine residues, mimicking such phosphates constitutively by incorporation of an aspartic acid can render the molecule to be constitutively active. In order to replace the tyrosine residue in Stat3 with aspartic acid, basic side directed mutagenesis approaches which are well known to the skilled artisan can be used. The present invention also relates to the expression of proteins that activate Stat3, such as IL-6. Expression of said protein components via gene therapy and resulting activation of Stat3 can be used in various diseases where a suppression of the immune response is desirable.

Another embodiment of the invention relates to the expression of the normal form of the Stat3 protein component. Despite the regulation of Stat3 signaling in a cell, elevating Stat3 protein levels in a cell can also increase its function thereby suppressing the immune response.

The nucleotide sequence to be expressed in a gene therapy approach has to be operatively linked to a promoter sequence. As it is known to the skilled artisan, enhancer/promoter sequences are essential for the expression of a given gene. Enhancer/promoter sequences also confer temporal and spatial regulation onto the expression pattern of a given gene.

Such enhancer/promoter sequences should be chosen dependent on the indicated disorder. In some cases tissue specific expression will be the preferred embodiment of the invention; in other cases systemic expression of the nucleotide sequence may be preferred. This decision will depend on the indicated disorder, and ultimately on the clinician. Expression specific to the tissue affected by the immunologic disorder can be conferred by enhancer/promoter sequences that are active only in that tissue. Combining the right promoter sequences with the gene to be expressed will require some experimentation involving standard techniques known to the skilled artisan. In other disorders, inducible expression of the immuno-suppressant protein, such as Stat3C, Stat3, or IL-6, may be indicated. As it is known to the skilled artisan, different enhancer/promoter sequences are active only in the absence and/or presence of a particular factor, which can be a metabolite, an anorganic molecule or a protein component.

5.4.2 Pharmacological Approaches

Immuno suppressant molecules can be delivered by administering the recombinant proteins. Recombinant Stat3, Stat3-C, or IL-6, respectively, can be synthesized and purified as fusion proteins by recombinant DNA techniques. Fusing a “peptide tag”, such as a polyhistidine tag, glutathione S-transferase (GST), or the E. coli maltose binding protein (MBP) to the angiogenic protein facilitates its purification. The fusion proteins can be synthesized in different host systems, such as, but not restricted to, bacteria, insects cells or mammalian cells. Methods of expressing said proteins in different systems and purifying them are described below.

In another embodiment of the invention, the proteins can be immuno-purified using antibodies specific to the respective protein. An examplary approach comprises covalently linking antibodies specific to the protein which is to be purified to a solid matrix. Protein extracts of the host cells expressing the desired protein are added to the matrix under conditions that allow binding of said protein to the matrix via non-covalent binding to the antibodies. After contaminants have been removed by washing under suitable conditions, the protein can be eluted.

The recombinant proteins can then be administered by the drug delivery system of choice dependent on whether systemic or local administration of the protein is preferred for the treatment or prevention, respectively, of the indicated disorder. Various delivery systems are known and are described below.

### 5.5 Methods for Identifying Immunologic Danger Signals

The invention relates to a method of identifying immunologic danger signals. Once those immunologic danger signals have been identified, they can be synthesized and administered to patients in order to augment the immune-response in various diseases.

In its preferred embodiment, the invention encompasses the identification of immunologic danger signals secreted by melanoma B16 cells that have been genetically engineered to express the dominant negative form of Stat3, Stat3beta. In a specific embodiment of the application, Stat3beta is expressed in melanoma B16 cells using the pIRES vector system (Clontech; Palo Alto, Calif.; Catlett-Falcone et al. 1999, Immunity 10:105-115). The nucleotide sequence encoding Stat3beta can be inserted into pIRES or any other vector system suitable for transfection of mammalian cells by standard molecular biology techniques. Likewise, the DNA can be transfected into the cells by standard techniques known to the skilled artisan. The supernatant of cells expressing Stat3beta comprises immunologic danger signals (see Example 2).

In order to identify the individual components of the supernatant that are responsible for the immunologic signaling activity of the supernatant, the components of the supernatant can be separated by standard biochemical techniques such as, but not limited to, gel-filtration chromatography or ion-exchange chromatography. These techniques are well known to the skilled artisan, and a minimum of experimentation will be required to determine the optimal conditions under which to purify individual components of the supernatant. After separation of the constituent components of the supernatant in individual components or fractions, said fractions are tested for their immunologic signaling effects on different immune cells, such as macrophages, T-cell and neutrophils. Dependent on the cell type, different assays can be employed in order to test the immunologic signaling activity of a given fraction. Those assays are described in the following subsections. Once positive fraction have been identified, the constituent components of a given fraction can be analyzed by techniques such as, but not limited to, SDS gel electrophoresis or mass spectrometry. In case the fraction of interest contains more than one component, the components of the fraction must be separated from each other and individually tested for their immunologic signaling activity in the respective assay. Again, standard biochemical techniques such as, but not limited to, gel-filtration chromatography or ion-exchange chromatography can be used for the isolation of the component of interest. Once a factor with immunologic signaling activity is isolated from the supernatant, its identity can be determined by using standard techniques such as micro-sequencing or mass-spectrometry.

In additional embodiments, the invention relates to the identification of immunologic danger signals released from cells other than melanoma B16 cells, but similarly expressing Stat3beta.

Furthermore, the invention encompasses a method of identifying immunologic danger signals released from cells, such as but not limited to, melanoma B16 cells, in which Stat3beta signaling is inhibited by specific antagonists of Stat3beta activity. Such antagonists comprise antisense nucleotide sequences specific to Stat3beta and ribozymes that act specifically on Stat3beta RNA.

5.5.1 Activating Immune Cells Such as Macrophages, T-Cells, and Neutrophils

Immunologic signaling activity can be tested either in cell culture on various types of cells of the immune system or in an animal model. Accordingly, the fractions, which are obtained from the supernatant as described above, are added either to cells in culture, such as cultures of macrophages, T-cells and neutrophils, or, alternatively, are injected into an animal, preferably a mouse. After a sufficient time period said cells are tested for immunologic activity. This can be accomplished for example by measuring the expression levels of markers of activation. In the case of macrophages such markers include, but are not limited to, the nitric oxide synthase, iNOS, and the chemokine RANTES. If the activation of T-cells is to be investigated, interferon-gamma (IFN-gamma) and interleukin-2 (IL-2) can be used as markers. Expression of the tumor necrosis factor alpha (TNF-alpha) can be used as a marker if neutrophils are used in this assay system. The length of the time period between stimulation and assay of expression of said markers may be changed and depends on the precise experimental conditions. A minimum of experimentation is necessary to establish the assay system to which the invention relates in such a way that it functions optimally. The levels of iNOS, RANTES, IFN-gamma, IL-2, and TNF-alpha can be determined by immunoblotting, Northern blotting, RNAse protection assays, immunocytochemistry or similar techniques well known to the skilled artisan. For any of those techniques probes specific to iNOS, RANTES, IFN-gamma, IL-2, and TNF-alpha, respectively, have to be employed. Such probes comprise antibodies and antisense RNA molecules. The detection of such probes is well established in the art.

If an animal model is employed, macrophages, T-cells and neutrophils can be isolated from the animal and subsequently analyzed or, alternatively, expression levels of iNOS, RANTES, IFN-gamma, IL-2, and TNF-alpha can be tested in situ by immunohistochemistry or in situ hybridization. For any of those techniques probes specific to iNOS, RANTES, IFN-gamma, IL-2, and TNF-alpha, respectively, have to be employed. Such probes comprise antibodies and antisense RNA molecules. The detection of such probes is well established in the art. Quantification and statistical analysis of the data is done by standard methods.

5.6 Therapeutic Methods for Use with the Invention

5.6.1 Recombinant DNA

In various embodiments of the invention, the Stat3 activity modulator comprises a protein which is encoded by a specific nucleotide sequence. In other embodiments of the invention, the pharmaceutical comprises a nucleotide sequence which is transcribed to generate a biologically active RNA molecule. In even other embodiments of the invention, the Stat3 activity modulator comprises a nucleotide sequence which is to be transcribed and translated. In either case, said nucleotide sequence is inserted into an expression vector for propagation and expression in recombinant cells or in cells of the host in the case of gene therapy.

An expression construct, as used herein, refers to a nucleotide sequence encoding the Stat3 activity modulator, which can be either an RNA molecule or a protein, operably linked to one or more regulatory regions or enhancer/promoter sequences which enables expression of the protein of the invention in an appropriate host cell. “Operably-linked” refers to an association in which the regulatory regions and the nucleotide sequence encoding the Stat3 activity modulator to be expressed are joined and positioned in such a way as to permit transcription, and ultimately, translation.

The regulatory regions necessary for transcription of the Stat3 activity modulator can be provided by the expression vector. In a compatible host-construct system, cellular transcriptional factors, such as RNA polymerase, will bind to the regulatory regions on the expression construct to effect transcription of the $tat3 activity modulator in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′-non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

Both constitutive and inducible regulatory regions may be used for expression of the Stat3 activity modulator. It may be desirable to use inducible promoters when the conditions optimal for growth of the host cells and the conditions for high level expression of the Stat3 activity modulator are different. Examples of useful regulatory regions are provided below (section 5.6.3).

In order to attach DNA sequences with regulatory functions, such as promoters, to the sequence encoding the Stat3 activity modulator or to insert the sequence encoding the Stat3 activity modulator into the cloning site of a vector, linkers or adapters providing the appropriate compatible restriction sites may be ligated to the ends of the cDNAs by techniques well known in the art (Wu et al., 1987, Methods in Enzymol 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

An expression construct comprising a sequence encoding the Stat3 activity modulator operably linked to regulatory regions (enhancer/promoter sequences) can be directly introduced into appropriate host cells for expression and production of the Stat3 activity modulator without further cloning. The expression constructs can also contain DNA sequences that facilitate integration of the sequence encoding the Stat3 activity modulator into the genome of the host cell, e.g., via homologous recombination. In this instance, it is not necessary to employ an expression vector comprising a replication origin suitable for appropriate host cells in order to propagate and express the protein of the invention in the host cells.

A variety of expression vectors may be used in the present invention which include, but are not limited to, plasmids, cosmids, phage, phagemids, or modified viruses. Typically, such expression vectors comprise a functional origin of replication for propagation of the vector in an appropriate host cell, one or more restriction endonuclease sites for insertion of the sequence encoding the Stat3 activity modulator, and one or more selection markers. The expression vector must be used with a compatible host cell which may be derived from a prokaryotic or an eukaryotic organism including but not limited to bacteria, yeasts, insects, mammals, and humans.

Vectors based on E. coli are the most popular and versatile systems for high level expression of foreign proteins (Makrides, 1996, Microbiol Rev, 60:512-538). Non-limiting examples of regulatory regions that can be used for expression in E. coli may include but not limited to lac, trp, lpp, phoA, recA, tac, T3, T7 and λP_{L} (Makrides, 1996, Microbiol Rev, 60:512-538). Non-limiting examples of prokaryotic expression vectors may include the λgt vector series such as λgt11 (Huynh et al., 1984 in “DNA Cloning Techniques”, Vol. I: A Practical Approach (D. Glover, ed.), pp. 49-78, IRL Press, Oxford), and the pET vector series (Studier et al., 1990, Methods Enzymol., 185:60-89). However, a potential drawback of a prokaryotic host-vector system is the inability to perform many of the post-translational processing of mammalian cells. Thus, an eukaryotic host-vector system is preferred, a mammalian host-vector system is more preferred, and a human host-vector system is the most preferred.

For expression of the Stat3 activity modulator in mammalian host cells, a variety of regulatory regions can be used, for example, the SV40 early and late promoters, the cytomegalovirus (CMV) immediate early promoter, and the Rous sarcoma virus long terminal repeat (RSV-LTR) promoter. Inducible promoters that may be useful in mammalian cells include but are not limited to those associated with the metallothionein II gene, mouse mammary tumor virus glucocorticoid responsive long terminal repeats (MMTV-LTR), β-interferon gene, and hsp70 gene (Williams et al., 1989, Cancer Res. 49:2735-42; Taylor et al., 1990, Mol. Cell Biol., 10:165-75). It may be advantageous to use heat shock promoters or stress promoters to drive expression of the Stat3 activity modulator in recombinant host cells.

In addition, the expression vector may contain selectable or screenable marker genes for initially isolating, identifying or tracking host cells that contain DNA encoding the elected Stat3 activity modulator. For long term, high yield production of the elected Stat3 activity modulator, stable expression in mammalian cells is preferred. A number of selection systems may be used for mammalian cells, including but not limited to the Herpes simplex virus thymidine kinase (Wigler et al., 1977; Cell 11:223), hypoxanthine-guanine phosphoribosyltransferase (Szybalski and Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for dihydrofolate reductase (dhfr), which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neomycin phosphotransferase (neo), which confers resistance to the aminoglycoside G-418 (Colberre-Garapin et al., 1981, J. Mol. Biol. 150:1); and hygromycin phosphotransferase (hyg), which confers resistance to hygromycin (Santerre et al., 1984, Gene 30:147). Other selectable markers, such as but not limited to histidinol and Zeocin™ can also be used.

5.6.2 Production of Recombinant Proteins

5.6.2.1 Peptide Tagging

If the Stat3 activity modulator is a protein (hereinafter: the protein of the invention), generating a fusion protein comprising a peptide tag can aid its purification. In various embodiments, such a fusion protein can be made by ligating the nucleotide sequence encoding the protein of the invention to the sequence encoding the peptide tag in the proper reading frame. If genomic sequences are used, care should be taken to ensure that the modified gene remains within the same translational reading frame, uninterrupted by translational stop signals and/or spurious messenger RNA splicing signals.

In a specific embodiment, the peptide tag is fused at its amino terminal to the carboxyl terminal of the protein of the invention. The precise site at which the fusion is made is not critical. The optimal site can be determined by routine experimentation.

A variety of peptide tags known in the art may be used in the modification of the protein of the invention, such as but not limited to the immunoglobulin constant regions, polyhistidine sequence (Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67:21-30), and various cellulose binding domains (U.S. Pat. Nos. 5,496,934; 5,202,247; 5,137,819; Tomme et al., 1994, Protein Eng. 7:117-123), etc. Other peptide tags are recognized by specific binding partners and thus facilitate isolation by affinity binding to the binding partner, which is preferably immobilized and/or on a solid support. As will be appreciated by those skilled in the art, many methods can be used to obtain the coding region of the above-mentioned peptide tags, including but not limited to, DNA cloning, DNA amplification, and synthetic methods. Some of the peptide tags and reagents for their detection and isolation are available commercially.

5.6.2.2 Expression Systems and Host Cells

Preferred mammalian host cells include but are not limited to those derived from humans, monkeys and rodents, (see, for example, Kriegler M. in “Gene Transfer and Expression: A Laboratory Manual”, New York, Freeman & Co. 1990), such as monkey kidney cell line transformed by SV40 (COS-7, ATCC CRL 1651); human embryonic kidney line (293, 293-EBNA, or 293 cells subcloned for growth in suspension culture, Graham et al., J. Gen. Virol., 36:59, 1977; baby hamster kidney cells (BHK, ATCC CCL 10); Chinese hamster ovary-cells-DHFR (CHO, Urlaub and Chasin. Proc. Natl. Acad. Sci. 77; 4216, 1980); mouse sertoli cells (Mather, Biol. Reprod. 23:243-251, 1980); mouse fibroblast cells (NIH-3T3), monkey kidney cells (CVI ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); and mouse mammary tumor cells (MMT 060562, ATCC CCL51).

A number of viral-based expression systems may also be utilized with mammalian cells to produce the Stat3 activity modulator. Vectors using DNA virus backbones have been derived from simian virus 40 (SV40) (Hamer et al., 1979, Cell 17:725), adenovirus (Van Doren et al., 1984, Mol Cell Biol 4:1653), adeno-associated virus (McLaughlin et al., 1988, J Virol 62:1963), and bovine papillomas virus (Zinn et al., 1982, Proc Natl Acad Sci 79:4897). In cases where an adenovirus is used as an expression vector, the donor DNA sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing heterologous products in infected hosts. (See e.g., Logan and Shenk, 1984, Proc. Natl. Acad. Sci. (USA) 81:3655-3659).

Other useful eukaryotic host-vector system may include yeast and insect systems. In yeast, a number of vectors containing constitutive or inducible promoters may be used with Saccharomyces cerevisiae (baker's yeast), Schizosaccharomyces pombe (fission yeast), Pichia pastoris, and Hansenula polymorpha (methylotropic yeasts). For a review see, Current Protocols in Molecular Biology, Vol. 2, 1988, Ed. Ausubel-et al., Greene Publish. Assoc. & Wiley Interscience, Ch. 13; Grant et al., 1987, Expression and Secretion Vectors for Yeast, in Methods in Enzymology, Eds. Wu & Grossman, 1987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II.

In an insect system, Autographa californica nuclear polyhidrosis virus (AcNPV) a baculovirus, can be used as a vector to express the protein of the invention in Spodoptera frugiperda cells. The sequences encoding the protein of the invention may be cloned into non-essential regions (for example the polyhedrin gene) of the virus and placed under control of an AcNPV promoter (for example the polyhedrin promoter). These recombinant viruses are then used to infect host cells in which the inserted DNA is expressed. (See e.g., Smith et al., 1983, J Virol 46:584; Smith, U.S. Pat. No. 4,215,051.)

Any of the cloning and expression vectors described herein may be synthesized and assembled from known DNA sequences by well known techniques in the art. The regulatory regions and enhancer elements can be of a variety of origins, both natural and synthetic. Some vectors and host cells may be obtained commercially. Non-limiting examples of useful vectors are described in Appendix 5 of Current Protocols in Molecular Biology, 1988, ed. Ausubel et al., Greene Publish. Assoc. & Wiley Interscience, which is incorporated herein by reference; and the catalogs of commercial suppliers such as Clontech Laboratories, Stratagene Inc., and Invitrogen, Inc.

Expression constructs containing cloned nucleotide sequence encoding the protein of the invention can be introduced into the host cell by a variety of techniques known in the art, including but not limited to, for prokaryotic cells, bacterial transformation (Hanahan, 1985, in DNA Cloning, A Practical Approach, 1: 109-136), and for eukaryotic cells, calcium phosphate mediated transfection (Wigler et al., 1977, Cell 11:223-232), liposome-mediated transfection (Schaefer-Ridder et al., 1982, Science 215:166-168), electroporation (Wolff et al., 1987, Proc Natl Acad Sci 84:3344), and microinjection (Cappechi, 1980, Cell 22:479-488).

For long term, high yield production of the properly processed protein of the invention, stable expression in mammalian cells is preferred. Cell lines that stably express protein of the invention may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression constrict confers resistance to the selection and optimally allows cells to stably integrate the expression construct into their chromosomes and to grow in culture and to be expanded into cell lines. Such cells can be cultured for a long period of time while-the protein of the invention is expressed continuously.

5.6.2.3 Protein Purification

Generally, the protein of the invention can be recovered and purified from recombinant cell cultures by known methods, including ammonium sulfate precipitation, acid extraction, anion or cation exchange chromatography, phosphocellulose chromatography, immunoaffinity chromatography, hydroxyapatite chromatography, and lectin chromatography. Before the protein of the invention can be purified, total protein has to be prepared from the cell culture. This procedure comprises collection, washing and lysis of said cells and is well known to the skilled artisan.

However, the invention provides methods for purification of the protein of the invention which are based on the properties of the peptide tag present on the protein of the invention. One approach is based on specific molecular interactions between a tag and its binding partner. The other approach relies on the immunospecific binding of an antibody to an epitope present on the tag or on the protein which is to be purified. The principle of affinity chromatography well known in the art is generally applicable to both of these approaches.

Described below are several methods based on specific molecular interactions of a tag and its binding partner.

A method that is generally applicable to purifying protein of the invention that are fused to the constant regions of immunoglobulin is protein A affinity chromatography, a technique that is well known in the art. Staphylococcus protein A is a 42 kD polypeptide that binds specifically to a region located between the second and third constant regions of heavy chain immunoglobulins. Because of the Fc domains of different classes, subclasses and species of immunoglobulins, affinity of protein A for human Fc regions is strong, but may vary with other species. Subclasses that are less preferred include human IgG-3, and most rat subclasses. For certain subclasses, protein G (of Streptococci) may be used in place of protein A in the purification. Protein-A sepharose (Pharmacia or Biorad) is a commonly used solid phase for affinity purification of antibodies, and can be used essentially in the same manner for the purification of the protein of the invention fused to an immunoglobulin Fc fragment. Bound protein of the invention can be eluted by various buffer systems known in the art, including a succession of citrate, acetate and glycine-HCL buffers which gradually lowers the pH. This method is less preferred if the recombinant cells also produce antibodies which will be copurified with the protein of the invention. See, for example, Langone, 1982, J. Immunol. meth. 51:3; Wilchek et al., 1982, Biochem. Intl. 4:629; Sjobring et al., 1991, J. Biol. Chem. 26:399; page 617-618, in Antibodies A Laboratory Manual, edited by Harlow and Lane, Cold Spring Harbor laboratory, 1988.

Alternatively, a polyhistidine tag may be used, in which ease, the protein of the invention can be purified by metal chelate chromatography. The polyhistidine tag, usually a sequence of six histidines, has a high affinity for divalent metal ions, such as nickel ions (Ni²⁺), which can be immobilized on a solid phase, such as nitrilotriacetic acid-matrices. Polyhistidine has a well characterized affinity for Ni²⁺-NTA-agarose, and can be eluted with either of two mild treatments: imidazole (0.1-0.2 M) will effectively compete with the resin for binding sites; or lowering the pH just below 6.0 will protonate the histidine sidechains and disrupt the binding. The purification method comprises loading the cell culture lysate onto the Ni²⁺-NTA-agarose column, washing the contaminants through, and eluting the protein of the invention with imidazole or weak acid. Ni²⁺-NTA-agarose can be obtained from commercial suppliers such as Sigma (St. Louis) and Qiagen. Antibodies that recognize the polyhistidine tag are also available which can be used to detect and quantitate the protein of the invention.

Another exemplary peptide tag that can be used is the glutathione-S-transferase (GST) sequence, originally cloned from the helminth, Schistosoma japonicum. In general, a protein of the invention-GST fusion expressed in a prokaryotic host cell, such as E. coli, can be purified from the cell culture lysate by absorption with glutathione agarose beads, followed by elution in the presence of free reduced glutathione at neutral pH. Since GST is known to form dimers under certain conditions, dimeric protein of the invention may be obtained. See, Smith, 1993, Methods Mol. Cell Bio. 4:220-229.

Another useful peptide tag that can be used is the maltose binding protein (MBP) of E. coli, which is encoded by the malE gene. The protein of the invention binds to amylose resin while contaminants are washed away. The bound protein of the invention-MBP fusion is eluted from the amylose resin by maltose. See, for example, Guan et al., 1987, Gene 67:21-30.

The second approach for purifying the protein of the invention is applicable to peptide tags that contain an epitope for which polyclonal or monoclonal antibodies are available. It is also applicable if polyclonal or monoclonal antibodies specific to the protein of the invention are available. Various methods known in the art for purification of protein by immunospecific binding, such as immunoaffinity chromatography, and immunoprecipitation, can be used. See, for example, Chapter 13 in Antibodies A Laboratory Manual, edited by Harlow and Lane, Cold Spring Harbor laboratory, 1988; and Chapter 8, Sections I and II, in Current Protocols in Immunology, ed. by Coligan et al., John Wiley, 1991; the disclosure of which are both incorporated by reference herein.

5.6.3 Gene Therapy Approaches

In a specific embodiment, nucleotide sequences encoding Stat3, Stat3beta, Stat3-C, IL-6 or nucleotide sequences encoding therapeutic RNA molecules, such as antisense RNA and ribozymes specific to Stat3, are administered to treat, or prevent-various diseases. These nucleotide sequences are collectively referred to as nucleotide sequences of the invention. Gene therapy refers to therapy performed by the administration to a subject of an expressed or expressible nucleotide sequence. In this embodiment of the invention, the nucleotide sequences produce their encoded protein or RNA molecule that mediates a therapeutic effect.

Any of the methods for gene therapy available in the art can be used according to the present invention. Exemplary methods are described below.

For general reviews of the method of gene therapy, see, Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; Morgan and Anderson, 1993, Ann. Rev. Biochem. 62:191-217; May, 1993, TIBTECH 1, 1(5):155-215. Methods commonly known in the art of recombinant DNA technology which cap be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a specific embodiment, nucleic acid molecules are used in which the nucleotide sequence of the invention is flanked by regions that promote homologous recombination at a desired site in the genome, thus providing for intrachromosomal expression of the nucleotide sequence of the invention (Koller and Smithies, 1989, Proc. Natl. Acad. Sci. USA 86:8932-8935; Zijistra et al., 1989, Nature 342:435-438).

In a specific embodiment, the nucleic acid sequences are directly administered in vivo, where it is expressed to produce the encoded product. This can be accomplished by any of numerous methods known in the art, for example by constructing them as part of an appropriate nucleic acid expression vector and administering the vector so that the nucleic acid sequences become intracellular. Gene therapy vectors can be administered by infection using defective or attenuated retrovirals or other viral vectors (see, e.g., U.S. Pat. No. 4,980,286); direct injection of naked DNA; use of microparticle bombardment (e.g., a gene gun; Biolistic, Dupont); coating with lipids or cell-surface receptors or transfecting agents; encapsulation in liposomes, microparticles, or microcapsules; administration in linkage to a peptide which is known to enter the nucleus; administration in linkage to a ligand subject to receptor-mediated endocytosis (see, e.g., Wu and Wu, 1987, J. Biol. Chem. 262:4429-4432) (which can be used to target cell types specifically expressing the receptors); etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; W092/20316; W093/14188, and WO 93/20221). Alternatively, the nucleic acid can be introduced intracellularly and incorporated within host cell DNA for expression by homologous recombination (Koller and Smithies, 1989, Proc. Natl. Acad. Sci. USA 86:8932-8935; Zijlstra et al., 1989, Nature 342:435-438).

In a specific embodiment, viral vectors that contain the nucleotide sequence of the invention are used. For example, a retroviral vector can be used (see Miller et al, 1993, Meth. Enzymol. 217:581-599). These retroviral vectors contain the components necessary for the correct packaging of the viral genome and integration into the host cell DNA. The nucleotide sequences of the invention to be used in gene therapy are cloned into one or more vectors, thereby facilitating delivery of the gene into a patient. More detail about retroviral vectors can be found in Boesen et al., 1994; Biotherapy 6:29 1-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clip. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

The following animal regulatory regions, which exhibit tissue specificity and have been utilized in transgenic animals, can be used for expression in a particular tissue type: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in the liver (Pinkert et al., 1987, Genes and Devel. 1:268-276), alpha-fetoprotein gene control region which is active in the liver (Kirumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1: 161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

5.6.4 Inhibitory Antisense, Ribozyme and Triple Helix Molecules

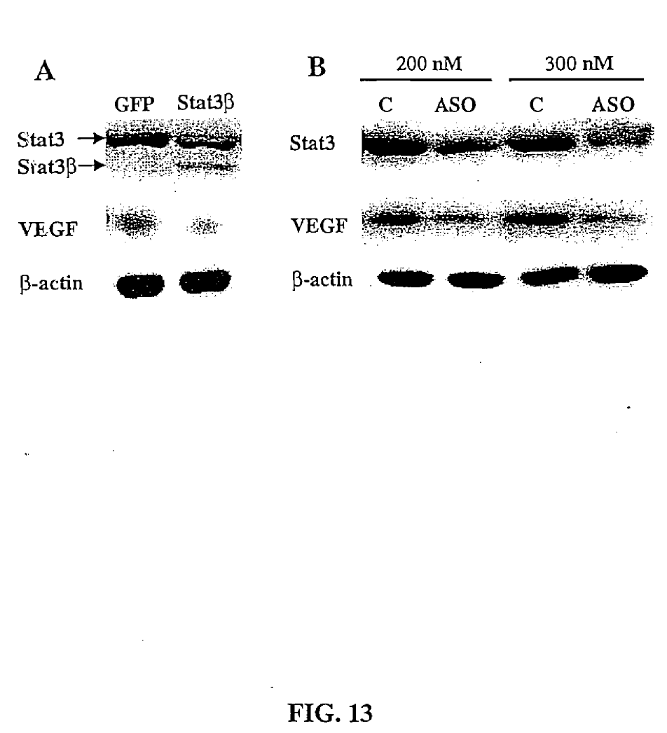

Among the compounds that may exhibit the ability to modulate the activity of Stat3 are antisense, ribozyme, and triple helix molecules. Techniques for the production and use of such molecules are well known to those of skill in the art. For example, antisense targeting Stat3 mRNA inhibits Stat3 signaling, as described in Section 8 (see FIGS. 12 and 13).

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to non-coding regions of the Stat3 gene could be used in an antisense approach to inhibit translation of endogenous Stat3 mRNA. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

In an embodiment of the present invention, oligonucleotides complementary to the nucleic acids encoding the Stat3 protein as indicated in SEQ ID. NO: 1.

Stat3 antisense molecules complementary to coding or non-coding regions may be used, members of both are well known in the art. Representative, non-limiting examples of Stat3 antisense molecules include the following: 5′-ACTCAAACTGCCCTCCTGCT-3′ (SEQ ID NO.: 6); 5′-TCTGAAGAAACTGCTTGATT-3′ (SEQ ID NO.: 7); 5′-GCCACAATCCGGGCAATCT-3′ (SEQ ID NO. 8); 5′-TGGCTGCAGTCTGTAGAAGG-3′ (SEQ ID NO. 9); 5′-TTTCTGTTCTAGATCCTGCA-3′ (SEQ ID NO.: 10); 5′-TAGTTGAAATCAAAGTCATC-3′ (SEQ ID NO.: 11); 5′-TTCCATTCAGATCTTGCATG-3′ (SEQ ID NO.: 12); 5′-TCTGTTCCAGCTGCTGCATC-3′ (SEQ ID NO.: 13); 5′-TCACTCACGATGCTTCTCCG-3′ (SEQ ID NO.: 14); 5′-GAGTTTTCTGCACGTACTCC-3′ (SEQ ID NO.: 15) (see, e.g., U.S. Pat. No. 6,159,694, issued Dec. 12, 2000, which is incorporated herein in its entirety).

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may, include other appended groups such as peptides (e.g., for targeting host cell receptors in vivo), or agents facilitating transport across the cell membrane (see, e.g., Letsinger, et al., 1989, Proc. Natl. Acad. Sci. U.S.A. 86, 6553-6556; Lemaitre, et al., 1987, Proc. Natl. Acad. Sci. 84, 648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6, 958-976) or intercalating agents (see, e.g., Zon, 1988, Pharm. Res. 5, 539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxamthine, xanthine, 4-acetylcytosine, 5-(carboxyhydroxylmethyl) uracil, 5-carboxymethylaminomethyl-w-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 5-methylaminomethyluracil, 5-methoxyaminomethyl-w-thiouracil, beta-D-mannsylqueosine, 5-methoxycarboxymetholuracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thioracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate (S-ODNs), a phosphorodithioate, a phosphoramidothioate, a phosphoramidate, a phosphordiamidate, a methylphosphonate, an alkyl phosphotriester, and a fornxacetal or analog thereof.

In yet another embodiment, the antisense oligonucleotide is an -anomeric oligonucleotide. An -anomeric oligonucleotide forms specific double-stranded hybrids with complementary RNA in which, contrary to the usual -units, the strands run parallel to each other (Gautier, et al., 1987, Nucl. Acids Res. 15, 6625-6641). The oligonucleotide is a 2-0-methylribonucleotide (Inoue, et al., 1987, Nucl. Acids Res. 15, 6131-6148), or a chimeric RNA-DNA analogue (Inoue, et al., 1987, FEBS Lett. 215, 327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein, et al. (1988, Nucl. Acids Res. 16, 3209), methylphosphonate oligonucleotides can be prepared by use of controlled pore glass polymer supports (Sarin, et al., 1988, Proc. Natl. Acad. Sci. U.S.A. 85, 7448-7451), etc.

While antisense nucleotides complementary to the target gene coding region sequence could be used, those complementary to the transcribed, untranslated region are most preferred.

In one embodiment of the present invention, gene expression downregulation is achieved because specific target mRNAs are digested by RNAse H after they have hybridized with the antisense phosphorothioate oligonucleotides (S-ODNs). Since no rules exist to predict which antisense S-ODNs will be more successful, the best strategy is completely empirical and consists of trying several antisense S-ODNs. Antisense phosphorothioate oligonucleotides (S-ODNs) will be designed to target specific regions of mRNAs of interest. Control S-ODNs consisting of scrambled sequences of the antisense SODNs will also be designed to assure identical nucleotide content and minimize differences potentially attributable to nucleic acid content. All S-ODNs will be synthesized by Oligos Etc. (Wilsonville, Oreg.). In order to test the effectiveness of the antisense molecules when applied to cells in culture, such as assays for research purposes or ex vivo gene therapy protocols, cells will be grown to 60-80% confluence on 100 mm tissue culture plates, rinsed with PBS and overlaid with lipofection mix consisting of 8 ml Opti-MEM, 52.8 1 Lipofectin, and a final concentration of 200 nM S-ODNs. Lipofections will be carried out using Lipofectin Reagent and Opti-MEM (Gibco BRL). Cells will be incubated in the presence of the lipofection mix for 5 hours. Following incubation the medium will be replaced with complete DMEM. Cells will be harvested at different time points postlipofection and protein levels will be analyzed by Western blot.

Antisense molecules should be targeted to cells that express the target gene, either directly to the subject in vivo or to cells in culture, such as in ex vivo gene therapy protocols. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol H promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290, 304-310), the promoter contained in the 3 long terminal repeat of Rous sarcoma virus (Yamamoto, et al., 1980, Cell 22, 787-797), the herpes thymidine kinase promoter (Wagner, et al., 1981, Proc. Natl. Acad. Sci. U.S.A. 78, 1441-1445), the regulatory sequences of the metallothionein gene (Brinster, et al., 1982, Nature 296, 39-42), etc. Any type of plasmid, cosmid, YAC or viral vector can be used to prepare the recombinant DNA construct which can be introduced directly into the tissue site. Alternatively, viral vectors can be used that selectively infect the desired tissue, in which case administration may be accomplished by another route (e.g., systemically).

Ribozyme molecules designed to catalytically cleave target gene mRNA transcripts can also be used to prevent translation of target gene mRNA and, therefore, expression of target gene product (see, e.g., PCT International Publication WO90/11364, published Oct. 4, 1990; Sarver, et al., 1990, Science 247, 1222-1225). In an embodiment of the present invention, oligonucleotides which hybridize to the Stat3 gene are designed to be complementary to the nucleic acids encoding the Stat3 protein (SEQ ID. NO: 2).

Ribozymes are enzymatic RNA molecules capable of catalyzing the specific cleavage of RNA. (For a review, see Rossi, 1994, Current Biology 4, 469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

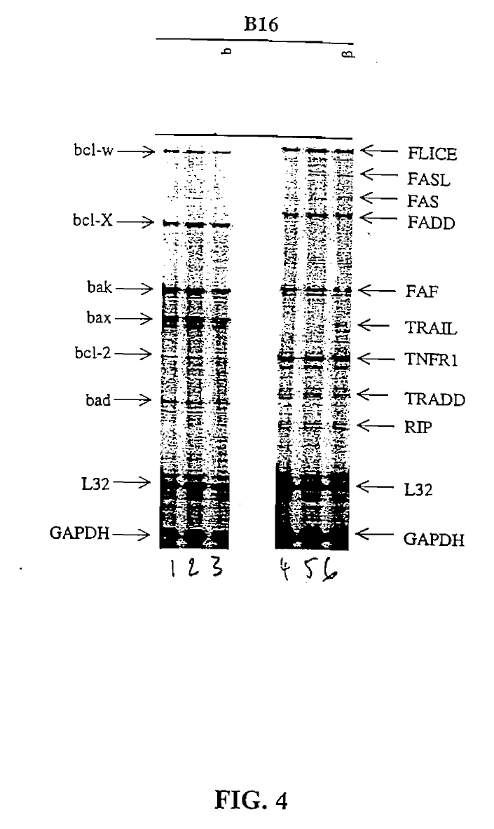

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff & Gerlach, 1988, Nature, 334, 585-591, which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224, 574-578; Zaug and Cech, 1986, Science, 231, 470-475; Zaug, et al., 1986, Nature, 324, 429-433; published International patent application No. WO 88/04300 by University Patents Inc.; Been & Cech, 1986, Cell, 47, 207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the target gene.

As in the antisense approach, the ribozymes can be composed of modified oligonucleotides (e.g:, for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies, et al., 1985, Nature 317, 230-234; Thomas & Capecchi, 1987, Cell 51, 503-512; Thompson, et al., 1989, Cell 5, 313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited modifications to ES (embryonic stem) cells can be used to generate animal offspring with an inactive target gene (e.g., see Thomas & Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Nucleic acid molecules to be used in triple helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity may, be introduced into cells via gene therapy methods such as those described, below, in Section 5.7.2 that do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

### 5.6.5 Antibodies to Stat3 and Derivatives

According to the invention, Stat3, its fragments or other derivatives, or analogs thereof, may be used as an immunogen to generate antibodies which immunospecifically bind such an immunogen.

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, 1 gM, IgD, IgA and IgY), class (e.g., IgG, IgG2, IgG3, IgG4, IgA, and IgA2) or subclass of immunoglobulin molecule. Examples of immunologically active portions of immunoglobulin molecules include F(ab) and F(ab′)2 fragments which can be generated by treating the antibody with an enzyme such as pepsin or papain. In a specific embodiment, antibodies to a human Stat3 protein are produced. In another embodiment, antibodies to a domain of Stat3 are produced.

Various procedures known in the art may be used for the production of polyclonal antibodies to Stat3 or derivative or analog. In a particular embodiment, rabbit polyclonal antibodies to an epitope of Stat3 encoded by a sequence or fragment of SEQ ID NO: 2, or a subsequence thereof, can be obtained. For the production of antibody, various host animals can be immunized by injection with the native Stat3, or a synthetic version, or derivative (e.g., fragment) thereof, including but not limited to rabbits, mice, rats, etc. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For preparation of monoclonal antibodies directed toward an Stat3 sequence or analog thereof, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for Stat3 together with genes from a human antibody molecule of appropriate biological activity can be used; such antibodies are within the scope of this invention.

According to the invention, techniques described for the production of single chain antibodies (U.S. Pat. No. 4,946,778) can be adapted to produce Stat3-specific single chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for Stat3s, derivatives, or analogs.

Antibody fragments which contain the idiotype of the molecule can be generated by known techniques. For example, such fragments include but are not limited to: the F(ab′)2 fragment which can be produced by pepsin digestion of the antibody molecule; the Fab′ fragments which can be generated by reducing the disulfide bridges of the F(ab′)2 fragment, the Fab fragments which can be generated by treating the antibody molecule with papain and a reducing agent, and Fv fragments.

In the production of antibodies, screening for the desired antibody can be accomplished by techniques known in the art, e.g. ELISA (enzyme-linked immunosorbent assay). For example, to select antibodies which recognize a specific domain of a STAT, e.g., the transcriptional activation domain, DNA binding domain, dimerization domain, SH2 domain, or SH3 domain, one may assay generated hybridomas for a product which binds to a Stat3 fragment containing such domain. For selection of an antibody that specifically binds a first Stat3 homolog but which does not specifically bind a different Stat3 homolog, one can select on the basis of positive binding to the first Stat3 homolog and a lack of binding to the second Stat3 homolog.

Antibodies specific to a domain of Stat3 are also provided, such as to a transcriptional activation domain, DNA binding domain, a dimerization domain, SH2 domain, SH3 domain.

The foregoing antibodies can be used in methods known in the art relating to the localization and activity of the Stat3 sequences of the invention, e.g., for imaging these proteins, measuring levels thereof in appropriate physiological samples, in diagnostic methods, etc.

In another embodiment of the invention (see infra), anti-Stat3 antibodies and fragments thereof containing the binding domain are used as therapeutics.

Anti-Stat3 antibodies can be obtained from Santa Cruz Biotechnology, Inc. (Santa Cruz, Calif.), Research Diagnostics, Inc. (Flanders, N.J.) or Zymed Laboratories (South San Francisco, Calif.). Alternatively, anti-Stat3 antibodies can be produced by any method known in the art for the synthesis of antibodies, in particular, by chemical synthesis or preferably, by recombinant expression techniques.

### 5.7 Target Diseases and Disorders

In one embodiment, Stat3 agonists may be used to stimulate angiogenesis for the treatment or prevention of ischemic diseases. Ischemia is caused by an impaired blood supply resulting from narrowed or blocked arteries that starve tissues of needed nutrients and oxygen. Thus, any condition which reduces the availability of nutrients or oxygen to a tissue, resulting in stress, damage, and finally, cell death, may be treated by the methods of the present invention. Ischemic disorders that may be treated by the methods described herein include, but are not limited to, coronary-atherosclerosis induced myocardial infarction and tissue ischemia in the lower extremities. In another embodiment, Stat3 agonists may be used to protect cardiac tissue from injury sustained during ischemia, infarction, inflammation, or trauma. These conditions arise from or include, but are not limited to stroke, vascular occlusion, prenatal or postnatal oxygen deprivation, suffocation, choking, near drowning, carbon monoxide poisoning, smoke inhalation, trauma, including surgery and radiotherapy, asphyxia, epilepsy, hypoglycemia, chronic obstructive pulmonary disease, emphysema, adult respiratory distress syndrome, hypotensive shock, septic shock, anaphylactic shock, insulin shock, sickle cell crisis, cardiac arrest, dysrhythmia, and nitrogen narcosis.

In another embodiment, autoimmune diseases that can be treated by the methods of the present invention include, but are not limited to, insulin dependent diabetes mellitus (i.e., IDDM, or autoimmune diabetes), multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, idiopathic thrombocytopenia purpura, rheumatoid arthritis, cirrhosis, pemphigus vulgaris, autoimmune infertility, Goodpasture's disease, bullous pemphigoid, discoid lupus, ulcerative colitis, and dense deposit disease. The diseases set forth above, as referred to herein, include those exhibited by animal models for such diseases, such as, for example non-obese diabetic (NOD) mice for IDDM and experimental autoimmune encephalomyelitis (EAE) mice for multiple sclerosis.

The methods of the present invention can be used to treat such autoimmune diseases by reducing or eliminating the immune response to the patient's own (self) tissue, or, alternatively, by reducing or eliminating a pre-existing autoimmune response directed at tissues or organs transplanted to replace self tissues or organs damaged by the autoimmune response.

Inflammation caused by infectious diseases may also be treated or prevented using the methods and compositions of the present invention. Such infectious diseases include those caused by intracellular pathogens such as viruses, bacteria, protozoans, and intracellular parasites. Viruses include, but are not limited to viral diseases such as those caused by hepatitis type B virus, parvoviruses, such as adeno-associated virus and cytomegalovirus, papovaviruses such as papilloma virus, polyoma viruses, and SV40, adenoviruses, herpes viruses such as herpes simplex type I (HSV-I), herpes simplex type II (HSV-II), and Epstein-Barr virus, poxviruses, such as variola (smallpox) and vaccinia virus, RNA viruses, including but not limited to human immunodeficiency virus type I (HIV-I), human immunodeficiency virus type II (HIV-II), human T-cell lymphotrophic virus type I (HTLV-I), and human T-cell lymphotrophic virus type II (HTLV-II); influenza virus, measles virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

In another embodiment, bacterial infections can be treated or prevented such as, but not limited to disorders caused by pathogenic bacteria including, but not limited to, Streptococcus pyogenes, Streptococcus pneumoniae, Neisseria gonorrhoea, Neisseria meningitides, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) fetus, Campylobacter jejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhiimurium, Salmonella typhii, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella Buis, Brucella melitensis, Mycoplasma spp., Rickettsia prowazeki, Rickettsia tsutsugumushi, Chlamydia spp., and Helicobacter pylori.

In another preferred embodiment, the methods can be used to treat or prevent infections caused by pathogenic protozoans such as, but not limited to, Entomoeba histolytica, Trichomonas tenas, Trichomonas hominis, Trichomonas vaginalis, Trypanosoma gambiense, Trypanosoma rhodesiense, Trypanosoma cruzi, Leishmania donovani, Leishmania tropica, Leishmania braziliensis, Pneumocystis pneumonia, Plasmodium vivax, Plasmodium falciparum, and Plasmodium malaria.

With respect to specific proliferative and oncogenic disease, the diseases that can be treated or prevented by the methods of the present invention include, but are not limited to: human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma. hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

Diseases and disorders involving a deficiency in cell proliferation or in which cell proliferation is desired for treatment or prevention, and that can be treated or prevented by antagonizing Stat3, include but are not limited to degenerative disorders, growth deficiencies, hypoproliferative disorders, physical trauma, lesions, and wounds; for example, to promote wound healing, or to promote regeneration in degenerated, lesioned or injured tissues, etc.

### 5.8 Pharmaceutical Formulations and Modes of Administration

In a preferred aspect, a pharmaceutical of the invention comprises a substantially purified protein, nucleic acid, or chemical (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Various delivery systems are known and can be used to administer the pharmaceutical of the invention, e.g., encapsulation in liposomes, microparticles, microcapsules, receptor-mediated endocytosis (see, e.g., Wu and Wu, 1987, J. Biol. Chem. 262:4429-4432), construction of a nucleic acid as part of a retroviral or other vector, etc. Methods of introduction include but are not limited to intradermal, intramuscular, intraperitoneal, intravenous, subcutaneous, intranasal, epidural, and oral routes. Nucleic acids and proteins of the invention may be administered by any convenient route, for example by infusion or bolus injection, by absorption through epithelial or mucocutaneous linings (e.g., oral mucosa, rectal and intestinal mucosa, etc.) and may be administered together with other biologically active agents such as chemotherapeutic agents. Administration can be systemic or local.

In a specific embodiment, it may be desirable to administer the nucleic acid or protein of the invention by injection, by means of a catheter, by means of a suppository, or by means of an implant, said implant being of a porous, non-porous, or gelatinous material, including a membrane, such as a sialastic membrane, or a fiber. Preferably, when administering a protein, including an antibody, of the invention, care must be taken to use materials to which the protein does not absorb.

In another embodiment, the compound or composition can be delivered in a vesicle, in particular a liposome (see Langer, 1990, Science 249:1527-1533; Treat et al., 1989, in Liposomes in the Therapy of Infectious Disease and Cancer, Lopez-Berestein and Fidler (eds.), Liss, New York, pp. 353-365; Lopez-Berestein, ibid., pp. 317-327; see generally, ibid.)

In yet another embodiment, the compound or composition can be delivered in a controlled release system. In one embodiment, a pump may be used (see Langer, supra; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, New York; Ranger and Peppas, 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

Other controlled release systems are discussed in the review by Langer, 1990, Science 249:1527-1533.

In a specific embodiment where a nucleic acid of the invention is administered, the nucleic acid can be administered in vivo to promote expression of its encoded protein or RNA molecule, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. 26. USA 88:1864-1868), etc. Alternatively, a nucleic acid can be introduced intracellularly and incorporated within host cell DNA for expression, by homologous recombination. For a more detailed description of gene therapy approaches, see section 5.6.3.

As alluded to above, the present invention also provides pharmaceutical compositions (pharmaceuticals of the invention). Such compositions comprise a therapeutically effective amount of a nucleic acid, chemical or protein of the invention, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the nucleic acid or protein of the invention, preferably in purified form, together with a suitable amount of carrier so as to provide the form for proper administration to the patient. The formulation should suit the mode of administration.

In a preferred embodiment, the pharmaceutical of the invention is formulated in accordance with routine procedures as a pharmaceutical composition adapted for intravenous administration to human beings. Typically, compositions for intravenous administration are solutions in sterile isotonic aqueous buffer. Where necessary, the pharmaceutical of the invention may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical of the invention is to be administered by infusion, it can be dispensed with an infusion bottle containing sterile pharmaceutical grade water or saline. Where the pharmaceutical of the invention is administered by injection, an ampoule of sterile water for injection or saline can be provided so that the ingredients may be mixed prior to administration.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The amount of the nucleic acid or protein of the invention which will be effective in the treatment or prevention of the indicated disease can be determined by standard clinical techniques. In addition, in vitro assays may optionally be employed to help identify optimal dosage ranges. The precise dose to be employed in the formulation will also depend on the route of administration, and the stage of indicated disease, and should be decided according to the judgment of the practitioner and each patient's circumstances. Effective doses may be extrapolated from dose-response curves derived from in vitro or animal model test systems.

The present invention may be better understood by reference to the following non limiting Examples, which are provided as exemplary of the invention. The following examples are presented in order to more fully illustrate the preferred embodiments of the invention. They should in no way be construed, however, as limiting the broad scope of the invention.

As is described hereinbelow, the studies that were performed by the inventors herein are standard, universally-accepted tests in animal models predictive of prophylactic and therapeutic benefit.

### 6. Example 1

### Overexpression of a Dominant-Negative Stat3 Variant Leads to Production of Soluble Factors that induce Cell Cycle Arrest and Apoptosis

In this example, gene therapy of B16 tumors with a dominant-negative Stat3 variant, designated Stat30, resulted in inhibition of tumor growth and tumor regression. Ten to fifteen percent of the tumor cells were transfected in vivo. The Stat3β-induced anti-tumor effect was associated with massive apoptosis of B16 tumor cells, indicating a potent bystander effect. Overexpression of Stat3β in B16 cells resulted in both apoptosis and cell cycle arrest. Importantly, apoptosis and cell cycle arrest also occurred in non-transfected B16 cells when they were co-cultured in separate chambers with Stat3β-transfected B16 cells, demonstrating that soluble factors mediated the bystander effect. RNase protection assays using multi-template probes specific for key physiologic regulators of apoptosis revealed that overexpression of Stat3β in B16 tumor cells induced the expression of the apoptotic effector, TRAIL. These in vitro results demonstrated that the observed in vivo bystander effect leading to tumor cell growth inhibition was mediated by soluble factors produced as a result of overexpression of Stat3β in the B16 tumor cells.

### 6.1 Introduction

Effective cancer gene therapies require the killing of genetically untransduced tumor cells (“bystander” cells) concomitant with genetically transduced tumor cells. Because transfection efficiency is a rate-limiting step for gene therapy, the efficacy of cancer gene therapy is enhanced by bystander effects.

It has recently been demonstrated that in vivo transgenic expression of Stat3β in murine B16 tumors results in tumor regression involving massive apoptosis of tumor cells despite relatively low transfection efficiencies (10 to 15%). To demonstrate the cellular and molecular mechanisms underlying the Stat3β-mediated bystander effects observed in vivo, this example describes in vitro studies. This example shows that inhibition of Stat3 activity in B16 cells leads to production of soluble factors that induce both apoptosis and cell cycle arrest. Consistent with the finding that soluble factors are involved in the bystander effects, induction of mRNA encoding the apoptosis effector, TRAIL, was detected in Stat3β-transfected B16 cells.

### 6.2 Materials and Methods

Plasmids. The bicistronic green fluorescent protein vector, pIRES-EGFP, was obtained from Clontech (Palo Alto, Calif.). Insertion of Stat3β cDNA into the pIRES-EGFP plasmid to construct pIRES-Stat3β was as described previously Catlett-Falcone et al., 1999, supra.

Cell culture and transfection. B16 murine melanoma cells were grown in RPMI 1640 containing 10% FBS. Transfections were performed using GenePORTER™ Transfection Reagent (Gene Therapy Systems, San Diego, Calif.) according to the manufacturer's instructions. To determine transfection efficiency, fluorescence intensities of B16 cells transfected with either pIRES-EGFP or pIRES-Stat3β were measured by FACS (Becton Dickinson Immunocytometry, CA) 24 h after transfection.

Nuclear extracts and EMSA. Nuclear extract preparation and EMSA analysis of STAT DNA-binding activity were performed as previously described Catlett-Falcone et al., 1999, supra.

Cell growth inhibition assay. Cells were plated at 1.7×105 cells/well in 6-well plates, followed by transfection with either pIRES-EGFP or pIRES-Stat3β plasmids 24 h later. Cells were harvested at 24 h, 48 h or 72 h to determine the numbers of live cells. Cell viability was determined by trypan blue exclusion.

Apoptosis assay. B16 cells transfected with pIRES-EGFP or pIRES-Stat3β were washed with CellScrub™ buffer (Gene Therapy Systems, San Diego, Calif.) 24 h after transfection. Apoptosis of transiently-transfected B16 cells was analyzed after staining with Annexin V-PE by two-color flow cytometry. Apoptosis of non-transfected tumor cells in the upper chambers of Transwell units was analyzed after staining with Annexin V-PE and VIA-PROBE™ 7-AAD (Pharmingen, San Diego, Calif.) by two-color flow cytometry.

Cell cycle analysis. Cell cycle analysis based on DNA content was performed. Cells were harvested, washed twice in PBS and resuspended in 70% ethanol on ice for at least 30 min. After centrifugation, cells were resuspended in 1 ml of propidium iodide staining solution (50 μg propidium iodide, 1 mg RNase A and 1 mg glucose per ml PBS) and incubated at room temperature for 30 min. The cells were analyzed by FACS using ModFit LT cell cycle analysis software (Verity Software, Topsham, Me.). Cells transfected with vectors encoding EGFP were fixed in 1 ml of 0.5% formaldehyde on ice for 10 min. before adding 70% ethanol.

Supernatant studies. The supernatants derived from either empty vector or Stat3β transfected B16 cells were collected at 12 h, 24 h, 36 h and 48 h after transfection and filtered through a 0.22 μm filter. Meanwhile, B16 cells were plated 5×10³/well in 96-well plate in triplicates. After cells were attached to the wells, 100 μl of fresh culture medium and 100 μl supernatant from each time point were added to each well. Cells in supernatants were cultured for 48 h before analysing. For direct cell number counting, cells were harvested and counted by trypan blue exclusion. For 3H-thymidine (3H-TdR) incorporation assay, 0.25 μci 3H-TdR was added to each well during the last 4 h of incubation, transferred to glassfiber filters by an automated cell harvester (Tomtec, Hamden, Conn.) and 3H-TdR incorporation was determined with a liquid scintillation β-counter (Pharmacia Wallac, Finland). For MTT assays, 5 μl MTT (10 mg/ml) was added to each well during the last 4 h of incubation. Cells were lysed in 100 μl DMSO and metabolic activity was quantified spectrophotometrically.

Co-culturing studies in Transwell units. B16 cells in the lower chambers of Transwell units were transfected with either pIRES-EGFP or pIRES-Stat3β plasmids. Five hours later, 5×10⁴ of either B16 or MethA cells were added to upper chambers. After 48 h co-culturing, cells in the upper chambers were harvested for both cell cycle analysis and apoptosis assays.

RNA isolation and RNase protection assay. Total RNA was isolated from 5.0×10⁶ cells by TRIzol reagent (Gibco BRL, Grand Island, N.Y.). RNase protection assays (RPA) were carried out using the PharMingen Riboquant mAPO-3 (TRAIL, FasL, CD95, and other death receptor associated genes) and mAPO-2 (Bcl-2 family members) multi-probe templates according to the manufacturer's protocol (PharMingen, San Diego, Calif.). Briefly, the multi-probe template was synthesized by in vitro transcription with incorporation of [³²P]-α UTP and purified on a G50 Sephadex column (5-Prime to 3-Prime, Boulder, Colo.). Specific activity was quantitated in a Beckman LS 6500 scintillation counter (Beckman, Schaumburg, Ill.). Purified probe (0.8-1.5×106 cpm/gl) was hybridized with 10 gg of total RNA for 16 h, followed by RNase digestion at 37° C. for 1 h. Protected RNA fragments were separated on a 5% polyacrylamide denaturing gel and quantified with Image Quant software (Molecular Dynamics, Sunnyvale, Calif.). RPAs are representative of three individual experiments.

### 6.3 Results

### Stat3β Overexpression in B16 Cells Disrupts Stat3 DNA-Binding Activity.

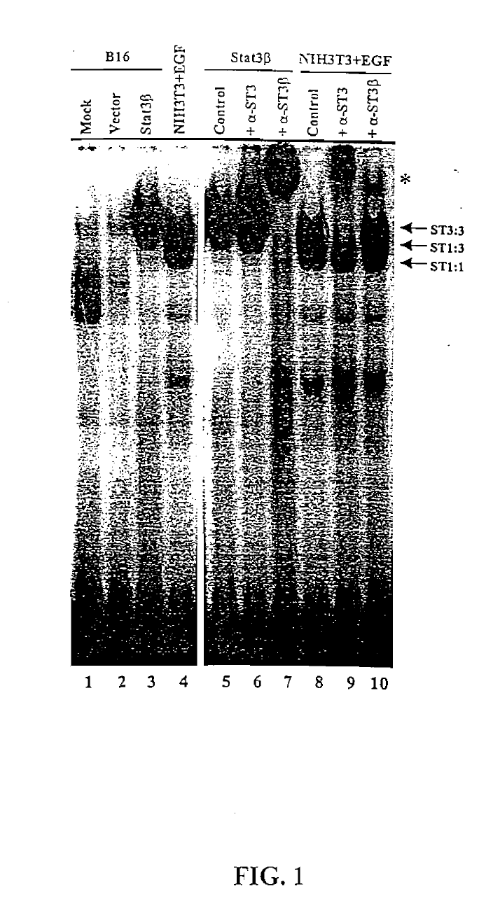

To show that Stat3β expression in transfected B16 cells inhibits endogenous Stat3 DNA-binding activity, EMSA with the ³²P-labeled hSIE probe that binds to Stat3 and Stat3β with high affinity was performed using nuclear extracts. FIG. 1 shows specific DNA-binding activities of endogenous Stat3 (lanes 1, 2) and ectopic Stat3β (lane 3). EGF-induced Stat3 binding activity in NIH3T3 (lane 4) was used as a positive control. By supershift analysis with antibody that recognizes Stat3 but not Stat3β or antibody that recognizes Stat3β but not Stat3, it was shown that there were Stat3-Stat3 homodimers in mock-transfected B16 cells and empty vector-transfected B16 cells. Overexpression of Stat3β in Stat3β-transfected B16 cells results in mostly Stat3β-Stat3β homodimer formation. These data suggest that Stat3β disrupts Stat3-specific gene regulation in B16 cells.

### Stat3β-Mediated B16 Cell Growth Inhibition Involves Both Cell Cycle Arrest and Apoptosis.

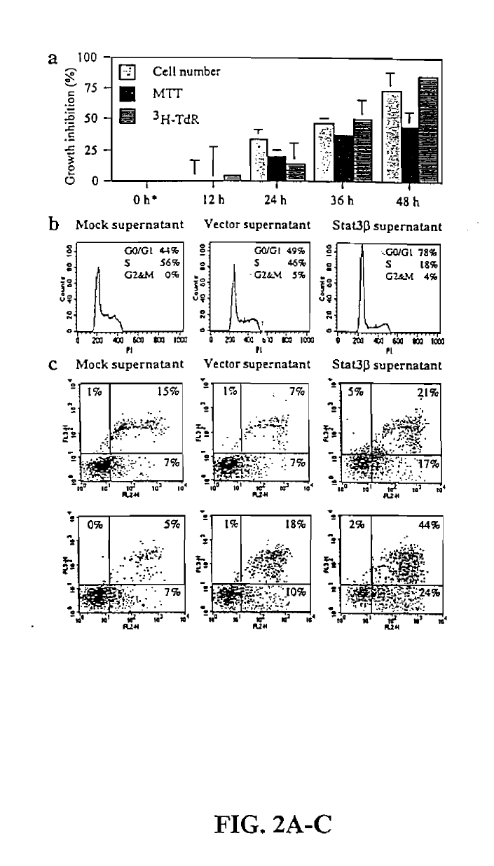

To show that transient transfection of pIRES-Stat3β leads to B16 cell growth inhibition, pIRES-EGFP or pIRES-Stat3β vectors were transfected into B16 cells, respectively. While their transfection efficiencies were similar within each experiment as determined by the percentage of cells that exhibit green fluorescence at 24 hours post transfection (by FACS analysis), the number of live B16 cells decreases dramatically 48 h later in the Stat3β-transfected population (FIG. 2A). To show that Stat3β-induced growth inhibition was mediated by cell cycle arrest and apoptosis, the effect of Stat3β on cell cycle progression and survival of B16 cells was examined. The cell cycle distributions of empty vector and Stat3β-transfected cells were shown in FIG. 2B. The Stat3β transfected B16 cells show progressive accumulation in G₀/G₁ phase, with concomitant decrease of the population in S and G₂/M phase. This G₀/G₁ phase arrest was observed as early as 24 hourspost transfection. At 48 hours post transfection with Stat3β vector, Annexin V-PE staining followed by FACS analysis to detect apoptotic activity was performed with transfected B16 cells. A high level of apoptosis in Stat3β transfected cells (75%) relative to empty vector transfected cells (25%) was observed, as shown in FIG. 2C. Increased levels of apoptosis as a result of Stat3β transfection was confirmed by confocal laser scanning microscope analysis using rhodamine-labelled TUNEL assay. This example demonstrates that Stat3β-mediated growth inhibition of B16 cells in vitro involves both cell cycle arrest and apoptosis.

### Stat3β Overexpression Leads to Production of Soluble Factors Capable of Inducing Both Cell Cycle Arrest and Apoptosis.

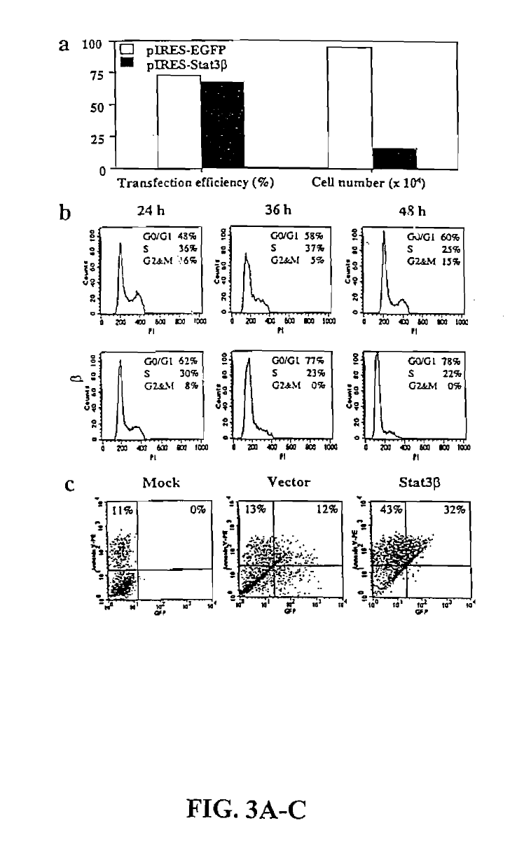

Many of the GFP-negative B16 cells (non-transfected) in the B16 cell culture transiently transfected with Stat3β also undergo apoptosis (FIG. 2C). This indicated that overexpression of Stat3β in B16 cells leads to bystander effects in vitro. To show that Stat3β-dependent bystander effects were not mediated by cell-cell contact, but were mediated via soluble factors, supernatants were collected 24 h, 36 h, 48 h after Stat3β vector transfection and subsequently used as conditioned medium for non-transfected B16 cells. Different assays for growth inhibition (cell number counts, MTT assays and 3H-TdR incorporation assays) were performed to show that the supernatants from Stat3β-transfected B16 cells inhibit the growth of non-transfected B16 cells. FIG. 3A shows that the conditioned media obtained from Stat3β-transfected B16 cells inhibit B16 cell growth, while that obtained from wild-type B16 cells or empty vector-transfected B16 cells does not. To rule out the possibility that Stat3β-induced growth inhibition of non-transfected tumor cells was due to apoptosis or stress in general, supernatant derived from UV-irradiated, apoptotic B16 cells was tested for its ability to inhibit B16 cell growth. Results show that supernatant derived from UV-irradiated, apoptotic B16 cells failed to induce any growth inhibition of B16 cells.

To show that soluble factor-induced growth inhibition was through apoptosis and cell cycle arrest, experiments using Transwell units were performed. A significant increase in the percentage of B16 cells arrested in G₀/G₁ was observed when cultured in conditioned medium derived from Stat3β-transfected B16 cells was compared to those cultured in conditioned media from mock or vector-transfected B16 cells. Furthermore, non-transfected B16 cells cultured in upper chambers in which the lower chambers contained Stat3β-transfected B16 cells undergo apoptosis as demonstrated by Annexin. V-PE and 7-AAD staining followed by FACS analysis (FIG. 3C). These Stat3β-induced soluble factors produced by transfected-B16 cells were also capable of inducing apoptosis of nontransfected Meth A cells (FIG. 3C).

### Expression of the Apoptosis Effector, TRAIL, was Induced in Stat3β-Transfected B16 Cells.

To demonstrate the identity of factors that cause apoptosis of untransfected tumor cells as a result of Stat3βexpression in B16 cells, RNase protection assays (RPAs) using multi-template probes were performed. Thirty hours after transfection, total RNA was isolated from various cell cultures and RPAs were carried out using probes specific for key physiologic regulators of apoptosis. An induction of TRAIL RNA expression in B16 cells as a result of Stat3β overexpression was detected (FIG. 4). This induction of TRAIL was specific, as none of the other genes examined was induced (FIG. 4).

### 6.4 Discussion

The potential of Stat3β gene therapy as an effective cancer therapeutic approach is supported by the finding that in vivo the number of dying tumor cells greatly exceeds the number of tumor cells transfected with Stat3β. In vitro results presented herein demonstrate that overexpression of Stat3β leads to apoptosis and cell cycle arrest of murine melanoma B16 cells. Importantly, disruption of Stat3 signaling in B16 cells also results in the production of soluble factors. The soluble factors were capable of inducing apoptosis and cell cycle arrest of non-transfected B16 tumor cells, showing that killing of bystander B16 tumor cells in vivo is mediated by one or more soluble factors.

Constitutively-activated Stat3 correlates with elevated levels of members of the Bcl-2 family of anti-apoptotic regulatory proteins, Bcl-X_{L} and Mcl-1 in human malignancies. Inhibition of Stat3 activity by Stat3β down regulates the expression of these anti-apoptotic proteins, resulting in apoptosis. In addition to inducing anti-apoptotic proteins, constitutive activation of Stat3 promotes the expression of proteins that were important for cell proliferation. In particular, cyclin D1, which controls progression from G1 to S phase, is elevated in cells expressing the constitutively-activated mutant form of Stat3, Stat3C, or endogenous Stat3 activated by the Src oncoprotein. Down-regulation of these and other anti-apoptotic and pro-proliferation proteins by Stat3β could, without being limited by theory, explain why overexpression of Stat3β in B16 tumor cells leads to both apoptosis and cell cycle arrest. Again, without being limited by theory, activated Stat3 could contribute to oncogenesis by preventing apoptosis and promoting proliferation by down regulating proapoptotic and anti-proliferative genes. This example shows that inhibition of Stat3 activity in B16 cells induces expression of the pro-apoptotic effector, TRAIL. TRAIL is a type II membrane protein but various cell types produce a soluble form of TRAIL.

Taken together, the experiments described in this example demonstrate a role for soluble factors produced by tumor cells in mediating Stat3β-dependent bystander effects, as a result of disrupting endogenous Stat3 activity.

### 7. Example 2

### Inhibition of Stat3 Signaling in B16 Cells Induces Secretion of Immunologic Danger Signals

This example shows that blocking Stat3 signaling induces the secretion of immunologic danger signals. B16 tumors treated in vivo with a Stat3 dominant-negative variant, Stat3β, become infiltrated with iNOS-positive macrophages and T cells. Inhibition of Stat3 signaling in B16 tumor cells results in secretion of soluble factors, which activate macrophages to produce additional inflammatory and tumoricidal mediators, including nitric oxide. Furthermore, transfection of B16 cells with the Stat3β gene upregulates expression of pro-inflammatory factors, including IL-6, IP-10, IFN-β and TNF-α, capable of stimulating nitric oxide production by macrophages. Significantly, this in vivo study demonstrates that expression of Stat3β in tumor cells leads to systemic activation of macrophages and T cells. This example shows that inhibition of Stat3 signaling can generate a cascade of immunologic danger signals important for activating immune responses, thus enhancing the utility of the present invention.

### 7.1 Introduction

This study demonstrates that Stat3βgene therapy of B16 tumors was accompanied by heavy infiltration of immune cells, including macrophages, neutrophils and T cells. Significantly, this example demonstrates that tumor-infiltrating macrophages after Stat3β gene therapy were strongly positive for iNOS expression, showing that the macrophages were activated in vivo. A critical role of iNOS induction in mediating the Stat3β-induced bystander effects in vivo was shown by detection of macrophage-stimulating soluble factors as a result of Stat3β expression in B16 tumor cells. These factors stimulate peritoneal macrophages to synthesize NO, which in turn has a strong cytostastic effect on B16 cells. Blockade of iNOS production, either in the presence of an iNOS inhibitor, NMA, or using iNOS deficient macrophages, abrogates soluble factor-induced, macrophage-mediated anti-B16 activity. Furthermore, our results demonstrate that inhibition of Stat3 signaling in B16 tumor cells results in elevated expression of IP-10, IL-6, TNF-α and IFN-β. Production of the soluble factors, including these pro-inflammatory cytokines and chemokines, in turn upregulates the expression of RANTES in macrophages and TNF-α in neutrophils. Thus, inhibition of Stat3 signaling results in. the induction of a cascade of immunologic danger signals, which are normally produced only during inflammation and infection. Activation of local inflammatory responses in the tumor microenvironment is known to be critical in stimulating antitumor T cells. This example shows that inhibiting Stat3 signaling in tumors, via activated innate immunity, leads to activation of T cells. This example thus provides support for an immunologic basis for the observed strong bystander effect that increases the antitumor efficacy of Stat3β gene therapy.

### 7.2 Methods

Tumor cells and supernatants. The B16 melanoma cells were cultured in RPMI medium with 10% FBS. Transfections were performed using GenePORTER™ Transfection. Reagent (Gene Therapy Systems, San Diego, Calif.) according to the manufacturer's instructions. To determine transfection efficiency, fluorescence intensities of B16 cells transfected with either pIRES-EGFP or pIRES-Stat3 were measured by FACS (Becton Dickinson Immunocytometry, CA) 24 h after transfection. Supernatants were collected at various time points as indicated in figures and figure legends. Supernatants were also collected from B16 cells treated UV-irradiation at various time points.

Mice. Six- to eight-week old female C57/B6 mice were obtained from the National Cancer Institute (Frederick, Md.). Cohorts of 3-5 mice per group were used for these experiments. To induce tumor, mice were shaved on the left flank and injected s.c. with 5×10⁵ of B16 cells in 100 μl of PBS. Gene therapy with Stat3β of established B16 tumors was described previously [Niu, 1999 #72].

Antibody staining of iNOS in B16 tumors. 3 μm paraffin sections were deparaffinized and endogenous peroxidase was blocked with 3% aqueous hydrogen peroxide. To minimize non-specific binding, the sections were incubated for 20 min in normal goat serum in PBS, followed by overnight incubation at 4° C. with rabbit anti-iNOS polyclonal antibody (Transduction Laboratories). After washing with PBS, sections were incubated for 2 min with diaminobenzidine tetrahydrochloride, rinsed with tap water and counterstained with modified Mayer's hematoxylin. Sections were dehydrated, cleared and mounted. Peritoneal macrophages and neutrophils.

Peritoneal macrophages were obtained and enriched. Neutrophils were obtained from peritoneal cavity 4 h after i.p. injection of 1 ml of 3% thioglycollate. The percentage of macrophages and neutrophils was estimated by morphological criteria using Giemsa staining (>98%). Macrophages were incubated for 48 h in conditioned medium containing 50% supernatants from either non-transfected, or pIRES-Stat3β or pIRES-EGFP transfected, or UV-irradiated B16 cells. Macrophage supernatants (0.1 ml) were collected and examined for nitric oxide accumulation using Griess reagent. Neutrophil supernatants were tested for TNF-α production using ELISA (R & D Systems, MN). MTT assay was performed to ensure that the viability of macrophages and neutrophils cultivated in different supernatants was not affected.

Macrophage-mediated antitumor cytotoxicity. Antitumor cytotoxic activity of macrophages against B16 cells was determined by inhibition of DNA synthesis. Briefly, peritoneal macrophages (1×10⁵) were incubated in 50% supernatants derived from either wild type, pIRES-Stat3β or pIRES-GFP transfected, or UV-irradiated B16 cells for 6 h. After replacing the supernatants with normal complete medium, B16 cells (1.0×10⁴/well) were added and co-cultured for 48 h with and without peritoneal macrophages. For some experiments, NOS inhibitor, N-monomethyl-L-arginine (NMA) (0.5 mM, Sigma) or H202 quencher, catalase (500 U/ml, Boehringer Mannheim, Indianapolis, Ind.) were added to macrophages before adding supernatant from Stat3β-transfected B16 cells. The co-cultured cells were pulsed with 3H-thymidine (3H-TdR) (0.25 μCi/well) during the last 6 h of incubation to estimate DNA synthesis. 3H-TdR incorporation was determined using a liquid scintillation β-counter (Pharmacia Wallac, Finland).

RNase protection assays. 10 μg of total RNA isolated from either pIRES-EGFP- or pIRES-Stat3 β-transfected B16 cells (36 h after transfection) was hybridized to multi-template probes from PharMingen (mCK-5, Top Panel; mCK-3, Lower Panel) that were labeled with ³²P-dUTP using in vitro transcription. The RNA encoded by GAPDH housekeeping gene was used to normalize the amounts of RNAs loaded in each lane. Similar protocols were used to determine RNA expression profiles of macrophages treated with supernatants derived from B16 cells transfected with either Stat3β or GFP vector. For the mCK-5, Top Panel; mCK-3 RPAs, RNAs prepared from mock transfected B16 cells and UV-irradiated, apoptotic B16 cells were also included to serve as negative controls. For macrophage RNA analysis, RNAs prepared from macrophages treated with supernatants from mock transfected and UV-irradiated B16 cells were also included.

### 7.3 Results

B16 Tumors Treated with Stat3b Gene Transfer Were Infiltrated with Immune Cells, Including iNOS-Positive Macrophages

The bystander effect was demonstrated by complete regression of tumors despite in vivo transduction of <15% of tumor cells. To show that immune cells have a role in the antitumor bystander effect, histochemical or immunohistochemical staining of B16 tumors treated with either pIRES-Stat3β or the control expression vector, pIRES-EGFP, was performed. Tissue sections from control vector-treated and Stat3β-treated tumors were stained with H&E and anti-iNOS antibodies. A dramatic increase in number of inflammatory cells, including neutrophils, macrophages, and T cells in Stat3β-treated, but not control vector-treated, B16 tumors was observed. These results demonstrated that Stat3β gene therapy in B16 tumors lead to induction of iNOS gene expression in the tumor-infiltrating macrophages.

Inhibition of Stat3 Signaling in B16 Cells Stimulates Production of Soluble Factors that Induce NO Production by Macrophage.

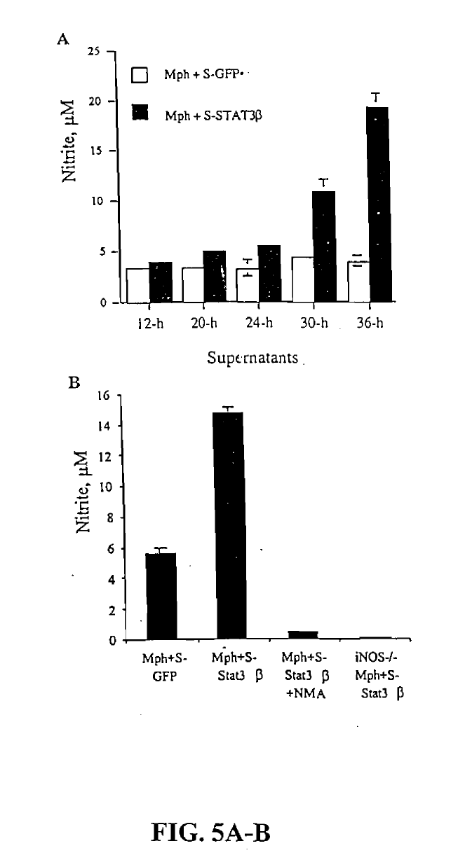

The observed inflammatory infiltrate in Stat3β-treated B16 tumors in vivo results from factors secreted by transfected B16 cells. To show that Stat3β-transfected B16 cells produce factors that contribute to activation of macrophages, the effects of the supernatants derived from B16 transfectants on NO production is demonstrated. Conditioned medium collected from macrophages treated with supernatant from Stat3β-transfected B16 cells, but not control B16 cells (mock transfected and GFP-control vector transfected), contained high levels of NO (FIG. 2). To rule out the possibility that production of soluble factors capable of activating macrophages produced by B16 tumor cells was due to apoptosis or stress in general, supernatant collected from UV-irradiated, apoptotic B16 cells was tested for its ability to induce macrophage NO production. In contrast to supernatant from Stat3β-transfected B16 cells, supernatant from UV-irradiated, apoptotic B16 cells fails to induce NO production (FIG. 5A). Results in FIG. 5B show that macrophage production of NO was iNOS-dependent, since blocking iNOS activity leads to abrogation of NO production.

### Nitric Oxide-Dependent Cytotoxic Activity Against B16 Tumor Cells by Soluble Factor Activated Macrophages.

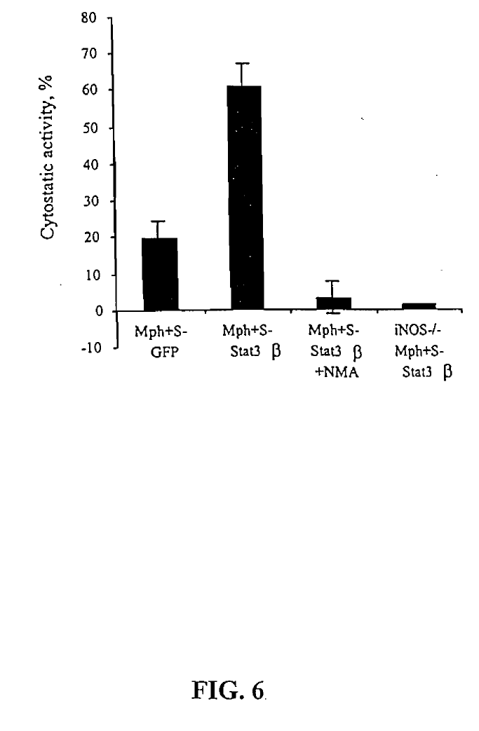

Nitric oxide was the key mediator of the tumoricidal activity of macrophages. This example demonstrates that soluble factor-induced macrophage NO production leads to cytotoxic activity against B16 tumor cells. 3H-thymidine incorporation to estimate DNA synthesis and cell proliferation was performed. In the results summarized in FIG. 6, supernatants derived from various B16 cells were removed from macrophages after 6 hours of incubation. B16 cells have little effect on the proliferation of non-transfected B16 cells, pre-incubation of macrophages in supernatant collected from pIRES-Stat3β-transfected, but not control vector-transfected B16 cells, induce strong cytostasis of non-transfected B16 cells (FIG. 6). Macrophage-mediated cytostasis of B16 cells was significantly blocked by a specific inhibitor of iNOS, NMA (FIG. 6). In contrast, addition of catalase, which inhibits H2O2 production, does not influence macrophage cytotoxic effects against B16 cells. Furthermore, soluble factor-induced macrophage cytostasis against B16 cells was abrogated when macrophages derived from iNOS knockout mice were used instead of those from wild-type mice (FIG. 6).

Blocking Stat3 Signaling in B16 Cells Elevates the Expression of Pro-Inflammatory Chemokines and Cytokines, which in Turn Activates Inflammatory Cells to Produce Additional Danger Signals.

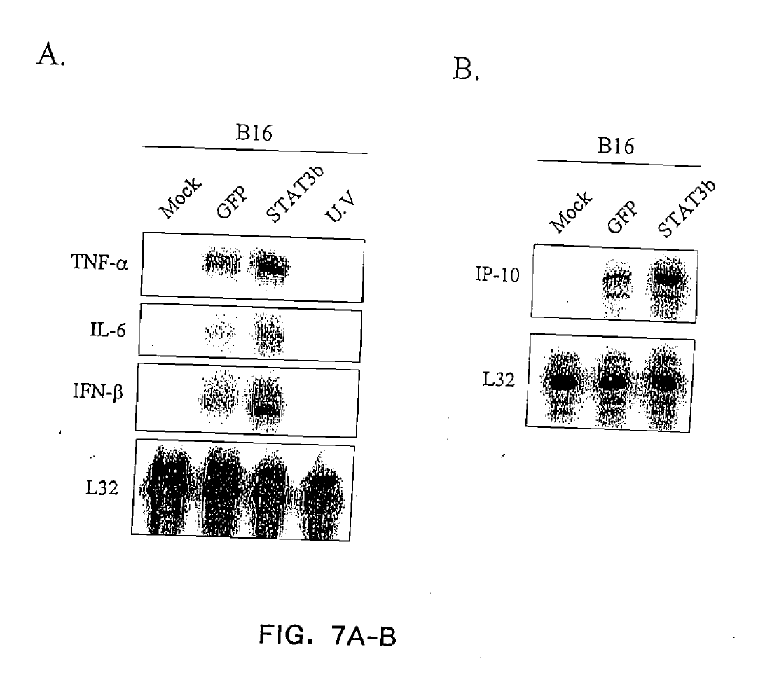

RNA expression profiles of a number of cytokines and chemokines in pIRES-Stat3β transfected B16 cells were determined. In addition, RNAs prepared from mock- and control vector-transfected, as well as UV-irradiated B16 cells were included as negative controls. Results from these RNase protection assays using multi-template RNA probes indicated that the expression levels of IFN-β, TNF-α, IL-6 and IP-10 mRNAs, but not IL-4 and IL-10, were elevated in Stat3β-transfected B16 cells in comparison with mock-transfected, control vector-transfected and UV-irradiated B16 cells (FIG. 7).

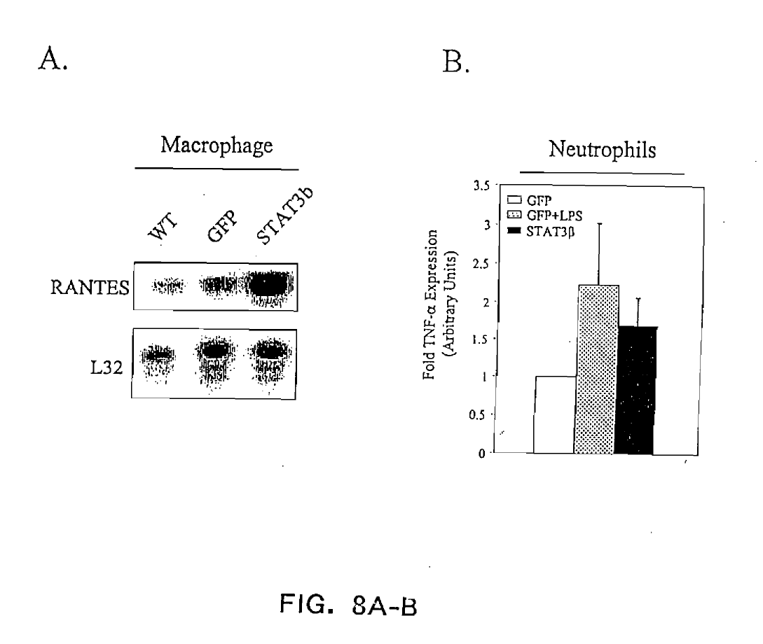

To show that these pro-inflammatory cytokines and chemokines participate in the activation of macrophages, macrophages were activated to produce NO by these cytokines in vitro. Peritoneal macrophages were able to synthesize NO when stimulated by IFN-β and TNF-α simultaneously. Moreover, supernatant derived from Stat3β-transfected B16 cells was capable of stimulating enhanced expression of RANTES by macrophages (FIG. 8A) and TNF-α by neutrophils (FIG. 8B). These chemokine and cytokines in turn further attract and activate macrophages.

### Inhibition of Stat3 Signaling in B16 Cells Leads to Systemic Activation of Macrophages T Cells.

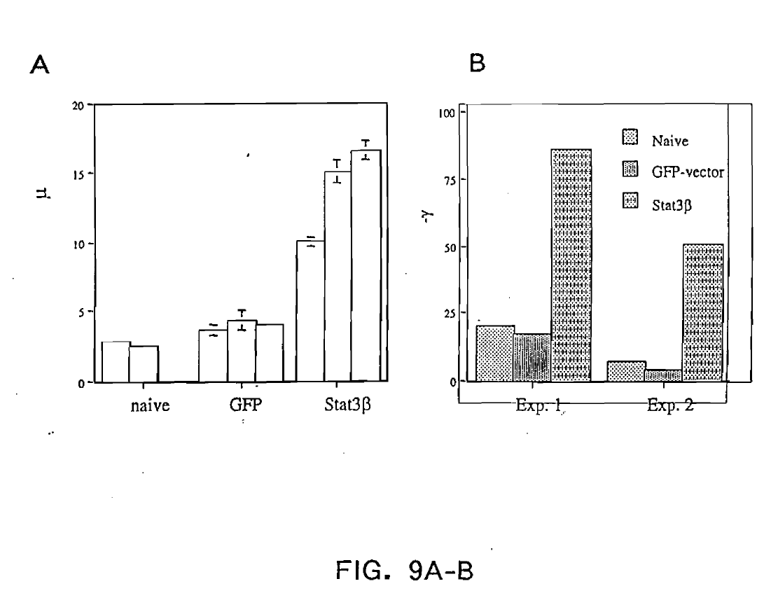

In addition to direct tumoricidal effect shown herein with production of NO by macrophages, innate immunity critically impacts the development of adaptive immune responses. To show that blocking Stat3 signaling in tumor cells causes macrophage and Th1 T cell activation, B16 cells transiently transfected with Stat3β were injected s.c. into mice. Compared to both naive mice and mice injected with control-vector transfected B16 cells, a clear induction of NO production by peritoneal macrophages was observed (FIG. 9A). Furthermore, a four-fold increase in IFN-γ production by lymphocytes derived from mice injected with Stat3β-transfected B16 cells was also detected (FIG. 9B).

### 7.4 Discussion

This example demonstrates that blocking Stat3 signaling in tumor cells results in secretion of immunologic danger signals, including pro-inflammatory cytokines and chemokines, which stimulate macrophages and neutrophils to produce additional inflammatory and tumoricidal mediators. Production of iNOS-dependent NO by macrophages stimulated by the soluble factors was shown to induce potent cytotoxic activity against non-transfected B16 tumor cells. Among the identified pro-inflammatory factors is also the T cell chemotractant, IP-10, which attracts T cells to the tumor site in vivo. This example shows that blocking Stat3 signaling in tumor cells causes a cascade of immune responses, leading to activation of T cells. Importantly, this example supports a mechanistic basis for the heavy infiltration of immune cells in tumors treated with Stat3β gene transfer and indicate a critical immune component to the potent bystander effect of gene therapy targeting Stat3 signaling in tumor cells.

This example has shown that targeting Stat3 signaling in tumor cells induces secretion of macrophage-activating factors that lead to production of iNOS-dependent NO, which in turn exerts potent, direct cytotoxic activity on tumor cells. Of the four pro-inflammatory cytokines and chemokines identified here, TNF-α and IFN-β were elicited upon ingestion of most microbes, showing the importance of these cytokines in activating iNOS for NO production. Induction of iNOS activity and subsequently the high-output pathway of NO production by macrophages under physiological conditions is only observed during inflammation and tissue damage due to viral or bacterial infections. Our demonstration that a direct antitumor effect is afforded by macrophage-produced, iNOS-dependent NO induced by inhibition of Stat3 signaling in tumor cells is therefore of great significance. Further, the importance of induction of iNOS and availability of NO at the tumor site is not limited to direct cytotoxic activity against tumor cells. There is a critical role of iNOS and NO in mediating T cell-dependent antitumor responses: GM-CSF vaccine-induced antitumor T cell immune response requires NO/iNOS, and IL-12-induces antitumor T-cell responses that were also iNOS/NO dependent.

Activation of innate immune responses demonstrated here can also be translated into stimulation of T cells. Among the pro-inflammatory factors resulting from inhibiting Stat3 signaling in B16 tumor cells were factors that can directly impact on tumor cell growth. TNF-α, for example, causes necrosis of tumor cells. Death of tumor cells in vivo itself promotes immunogenicity, because the release of tumor antigens under inflammatory conditions allows cross-priming of antigen-presenting cells, from which a tumor-specific T cell immunity can be elicited [Huang et al., 1994, Science 264:961-965; Huang et al., 1994, Ciba Found. Symp. 187:229-240; Dranoff et al., 1993, Proc. Natl. Acad. Sci. USA 90:3539-3543; Levitsky et al., 1994, J. Exp. Med. 179:1215-1224; Banchereau and Steinman, 1998, Nature 392:245-252; Maass et al., 1995, Int. J. Immunopharmacol. 17: 65-73; Maass et al., 1995, Proc. Natl. Acad. Sci. USA 92:5540-5544; Cayeux et al., 1997, Eur. J. Immunol. 27:1657-1662; Cayeux et al., 1997, J. Immunol. 158:2834-2841]. Targeting Stat3 signaling may generate tumor-specific T cell immunity.

Constitutive activation of Stat3 contributes to oncogenesis by helping tumor cells evade immune surveillance. The ability of normal cells to produce immunologic danger signals during infection and tissue destruction is well known, and Stat3 in hematopoietic cells is the regulator of macrophage activation. Stat3 mediates immune suppression by IL-10 signaling, which antagonizes the production of inflammatory cytokines such as TNF-α, IL-1 and IL-6, and suppresses iNOS activity. Blockade of Stat3 signaling, as in Stat3−/−macrophages, severely impairs the inhibitory activity of IL-10 on production of inflammatory cytokines. As a result, mice with Stat3−/− macrophage and neutrophils were highly susceptible to endotoxin shock, with increased production of TNF-α, IL-1, IL-6 and IFN-γ, and showed an enhanced T-helper 1 cell activity. Furthermore, Stat3−/− macrophages display increased expression of MHC class II and B7-1 molecules, thus Stat3 signaling suppress macrophage activation. Our present examples show a novel role for Stat3 signaling in nonhematopoietic tumor cells in blocking release of danger signals that activate a cascade of immune responses. As such, Stat3 activation in tumor cells may serve to cloak them from immune surveillance.

A critical role for Stat3 signaling in suppressing immune responses in normal nonhematopoietic cells during wound healing has also been suggested. Inflammatory cytokines and immune mediators, including TNF-α, IL-1, M-6 and NO, were in reduced amounts in naturally healing wounds compared to non-healing wounds [Trengove et al., 2000, Wound Repair Regen. 8: 13-25; Cao et al., 2000, Am. J. Sports Med. 28:176-182]. In the absence of Stat3 signaling, as shown in mice with epidermal and keratinocytes that lack functional Stat3, pronounced inflammatory infiltration is observed throughout the dermis while wound healing is impaired [Sano et al., 1999, EMBO J. 18:4657-4668]. Complementary to these findings were our current results in which blocking Stat3 signaling in tumor cells leads to upregulation of inflammatory factors, including production of TNF-α, IFN-β, IP-10, IL-6, and subsequent production of iNOS-dependent NO, RANTES by macrophages and TNF-α by neutrophils. Collectively, our results, together with recent studies using Stat3−/− inflammatory cells and skin cells [Takeda et al., 1999, Immunity 10:39-49; Sano et al., 1999, EMBO J. 18:4657-4668], suggest that Stat3 signaling down-regulates immunologic danger signals. Furthermore, constitutive activation of Stat3 may promote tumorigenesis by suppressing danger signals, thereby helping tumor cells escape immune recognition of their antigens.

### 8. Example 3

### Stat3 Signaling in Tumor Cells Promotes Angiogenesis Through Upregulation of VEGF

Stat3 signaling is required for cell transformation by v-Src. Activity of Src tyrosine kinase has been shown to regulate the expression of VEGF, a potent stimulator of angiogenesis, which is crucial for tumor growth and metastasis formation. In this third example, it was shown that blocking Stat3 signaling inhibits v-Src-mediated VEGF upregulation, and expression of constitutively-activated Stat3 increases the production of VEGF in fibroblasts. In tumor cells, blocking Stat3 signaling inhibits transcriptional activity of the VEGF promoter and down-regulates expression of the endogeneous VEGF gene. This example shows that constitutive Stat3 signaling upregulates VEGF expression, which in turn induces angiogenesis. Therefore, in the present invention, inhibition of Stat3 signaling inhibits angiogenesis mediated by downregulation of VEGF expression. And activation of Stat3 signaling promotes angiogenesis mediated by upregulation of VEGF expression.

### 8.1 Introduction

Angiogenesis plays a critical role in a wide variety of disorders, such as ischemic diseases and proliferative angiopathies with neovascularization. Vascular endothelial growth factor (VEGF) has been shown to be a potent endothelial cell-specific mitogen that stimulates angiogenesis. An essential role of VEGF in tumorigenesis has been shown when systemic treatment of tumor-bearing animals with a neutralizing antibody to VEGF inhibits tumor growth, which correlates with reduced tumor vascularity.

Constitutive activation of Stat3 in numerous human solid tumors was caused by deregulated activities of c-Src tyrosine kinase. In hemotopoietic malignancies, such as multiple myeloma, Stat3 was constitutively activated by IL-6 mediated signaling. Because Src tyrosine kinase activity and IL-6 mediated signaling can lead to both Stat3 activation and VEGF upregulation in tumor cells, it is demonstrated herein that Stat3 regulates VEGF expression in tumor cells. This example shows that Stat3 signaling was required for Src-induced VEGF upregulation and that Stat3 activity induces VEGF expression in tumor cells and in fibroblasts, showing that Stat3 plays an important role in VEGF expression and thus in angiogenesis.

### 8.2 Results

Stat3 Signaling was Required for v-Src-Induced VEGF Upregulation.

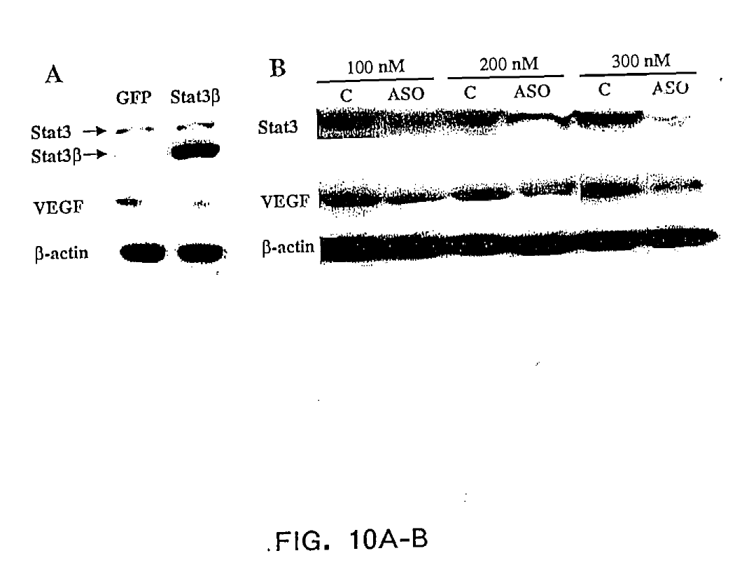

Src tyrosine kinase activity upregulates VEGF expression. Because Src-induced transformation requires Stat3 signaling, this example demonstrates that Stat3 is a requisite intermediary step for VEGF upregulation by Src activity. NIH3T3 fibroblasts transformed by v-Src were transiently transfected with a dominant-negative variant of Stat3, Stat3β. The transfection efficiency was approximately 40% based on the number of cells that were fluorescent due to the presence of GFP. The presence of Stat3β in the cells was accompanied by loss of Stat3 DNA binding activity as shown by an EMSA (FIG. 10A). Forty-eight hours later, VEGF expression in v-Src-NIH3T3 fibroblasts with or without Stat3β expression was compared at both RNA and protein levels. FIG. 10B shows that blocking Stat3 signaling in v-SrcNIH3T3 fibroblasts inhibits VEGF. Anti-sense oligonucleotides against Stat3 as well as control oligonucleotides were also transfected into v-Src-NIH3T3 cells. A reduction of endogenous Stat3 protein as a result of Stat3 anti-sense oligonucleotide also caused inhibition of VEGF expression.

Expression of a Constitutively-Activated Mutant form of Stat3 in Fibroblasts Stimulate the Production of VEGF

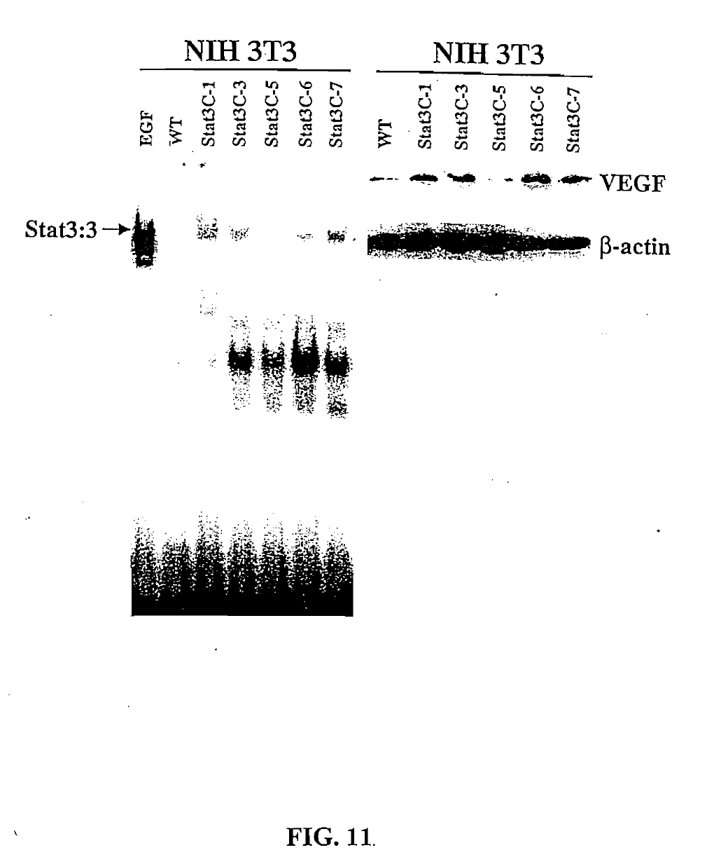

It was also shown herein that persistent Stat3 signaling by itself can upregulate VEGF expression. Transfection of a mutant form of Stat3 that was constitutively activated, Stat3-C (Bromberg et al., 1999, Cell 98:295-303), led to increased Stat3 DNA binding activity in several clones of NIH3T3 cells, which correlated with increased expression of VEGF at both protein and RNA levels (FIG. 11).

### Blocking Stat3 Signaling in Tumor Cells Inhibits VEGF Promoter Activity.

To show that transcriptional activity of VEGF promoter was regulated by the endogeneous Stat3 activity in tumor cells, B16 murine melanoma and SCK murine tumor cells were transiently transfected with Stat3β and a reporter construct containing luciferase cDNA under the control of the VEGF promoter. A plasmid construct containing the luciferase cDNA in the absence of the VEGF promoter was also transfected into 3T3 fibroblasts. Both of B16 and SCK tumor cells harbor constitutively activated Stat3. In the absence of Stat3β, VEGF promoter activity was readily detectable in both tumor cells as indicated by the high expression levels of luciferase protein. However, cotransfection with Stat3β, but not the control vector, greatly inhibited the transcriptional activity of the VEGF promoter. The inhibitory effect of Stat3β on the transcriptional activity of the VEGF promoter was also observed in the tumor cells transfected with Stat3 anti-sense oligonucleotides (FIG. 12A-B). It was also shown that blocking Stat3 signaling in tumor cells inhibits the expression of the endogeneous VEGF gene. B16 tumor cells were transiently transfected with Stat3β and the expression of endogeneous VEGF gene was determined at the RNA and protein levels. As shown in FIG. 13, inhibition of constitutive activation of Stat3 in tumor cells downregulates expression of the endogeneous VEGF gene.

### 8.3 Discussion

This example clearly shows that Stat3 is a requisite intermediary in the v-Src-induced VEGF expression, showing that Stat3 is an important regulator of VEGF-mediated angiogenesis. The present example establishes that constitutive signaling of Stat3 upregulates VEGF expression. Thus, a novel role of constitutive activation of Stat3 as an angiogenic regulator is shown.

Blocking Stat3 signaling, either by a Stat3 dominant-negative variant or antisense oligos, leads to antiangiogenesis via down regulation of VEGF, thus adding a new dimension to the therapeutic effect of anti-Stat3 signaling.

In addition to antiangiogenesis, downregulation of VEGF may also contribute to increased immune responses associated with inhibition of Stat3 signaling in tumor cells. VEGF produced by tumor cells have been shown to inhibit the functional maturation of dendritic cells, the most potent antigen presenting cells. Dendritic cells incubated with tumor cells in the presence of Stat3 antisense oligos, but not control oligos, undergo normal functional maturation.

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein, including patent applications, patents, and other publications, are incorporated by reference herein in their entireties for all purposes.

1. A method for inhibiting angiogenesis comprising administering to an individual in need of treatment an effective amount of a compound that inhibits the expression of human Stat3.

2. The method of claim 1, wherein said compound is an antisense oligonucleotide.

3. The method of claim 2, wherein said antisense oligonucleotide is an antisense RNA oligonucleotide.

4. The method of claim 2, wherein said antisense oligonucleotide is an antisense DNA oligonucleotide.

5. The method of claim 2, wherein said antisense oligonucleotide comprises a modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a fornxacetal backbone.

6. The method of claim 2, wherein said antisense oligonucleotide comprises a modified sugar moiety selected from the group consisting of arabinose, 2-fluoroarabinose, xylulose, and hexose.

7. The method of claim 2, wherein said antisense oligonucleotide comprises a modified base selected from the group consisting of 5-fluorouracil, 5-bromouracil, 5-chlorouracil, 5-iodouracil, hypoxamthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 5-methylaminomethyluracil, 5-methoxyaminomethyl-2-thiouracil, beta-D-mannsylqueosine, 5-methoxycarboxymetholuracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thioracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

8. The method of claim 2, wherein said antisense oligonucleotide is conjugated to a peptide, hybridization triggered cross-linking agent, transport agent, or hybridization-triggered cleavage agent.

9. The method of claim 2, wherein said compound is a recombinant DNA construct comprising a promoter operably linked to a nucleic acid encoding said antisense oligonucleotide.

10. The method of claim 1, wherein said compound is a ribozyme.

11. The method of claim 10, wherein said compound is a recombinant DNA construct comprising a promoter operably linked to a nucleic acid encoding said ribozyme.

12. A method for treating a proliferative angiopathy with neovascularization in an individual, said method comprising administering to said individual an effective amount of a compound that inhibits the expression of human Stat3.

13. The method of claim 12, wherein said compound is an antisense oligonucleotide.

14. The method of claim 13, wherein said antisense oligonucleotide is an antisense RNA oligonucleotide.

15. The method of claim 13, wherein said antisense oligonucleotide is an antisense DNA oligonucleotide.

16. The method of claim 12, wherein said proliferative angiopathy with neovascularization is a diabetic microangiopathy.

17. The method of claim 13, wherein said compound is a recombinant DNA construct comprising a promoter operably linked to a nucleic acid encoding said antisense oligonucleotide.

18. The method of claim 12, wherein said compound is a ribozyme.

19. The method of claim 18, wherein said compound is a recombinant DNA construct comprising a promoter operably linked to a nucleic acid encoding said ribozyme.
